(19) United States
(12) Patent Application Publication
(10) Pub. No.: US 2023/0129340 A1
(43) Pub. Date: Apr. 27, 2023

(54) METHOD FOR PRODUCING MULTISPECIFIC ANTIBODIES

(71) Applicant: Hoffmann-La Roche Inc., Little Falls, NJ (US)

(72) Inventors: Stefan SEEBER, Sindelsdorf (DE); Ulrich GOEPFERT, Penzberg (DE); Andrea OSTERLEHNER, Penzberg (DE); Hubert KETTENBERGER, Muenchen (DE); Wolfgang PAUL, Penzberg (DE)

(73) Assignee: Hoffmann-La Roche Inc., Little Falls, NJ (US)

(21) Appl. No.: 17/804,072

(22) Filed: May 25, 2022

Related U.S. Application Data
(63) Continuation of application No. 16/560,375, filed on Sep. 4, 2019, now abandoned.
Application No. 16/560,375 is a continuation of application No. PCT/EP2018/055532, filed on Mar. 7, 2018.

(30) Foreign Application Priority Data
Mar. 10, 2017 (EP) 17160415.0

(51) Int. Cl. C07K 16/28; C07K 16/18; C07K 16/46; C07K 16/00; C07K 16/40

(52) U.S. Cl. CPC C07K 16/2881; C07K 16/18; C07K 16/468; C07K 16/00; C07K 16/2818; C07K 16/2878; C07K 16/40; C07K 2317/31; C07K 2317/35

(57) ABSTRACT

Herein are provided a method for producing a multispecific antibody comprising the steps of providing a mammalian cell expressing the antibody, transfecting said mammalian cell with an expression vector comprising an expression cassette encoding a polypeptide of the antibody that has a domain crossover, cultivating the transfected cell and recovering the antibody from the cell or the cultivation medium and thereby producing the multispecific antibody.

### CROSS-REFERENCE TO RELATED APPLICATION

This application is a Continuation of U.S. patent application Ser. No. 16/560,375 filed Sep. 4, 2019, which is a Continuation of International Patent Application No. PCT/EP2018/055532 filed Mar. 7, 2018, which claims benefit of priority European Patent Application No. 17160415.0 filed Mar. 10, 2017, each of which is incorporated herein by reference in its entirety.

### SEQUENCE LISTING

This application contains a Sequence Listing which has been submitted electronically in ASCII format and is hereby incorporated by reference in its entirety. Said ASCII copy, created on May 25, 2022, is named P34166-US-1_SeqList.txt and is 853 bytes in size.

### FIELD OF THE INVENTION

The present invention relates to the production of multispecific antibodies especially to such multispecific antibodies comprising a domain crossover in one of their chains. In the method as reported herein the expression yield of a recombinant mammalian cell secreting the multispecific antibody is improved by the introduction of an additional expression cassette for a domain exchanged chain in an already transfected or transduced cell.

### BACKGROUND

U.S. Pat. No. 5,958,727 describes a method of producing a polypeptide, comprising cultivating a mutant cell under conditions conducive for production of the polypeptide, wherein the mutant cell is related to a parent cell, which comprises a first DNA sequence encoding the polypeptide, by the introduction of a nucleic acid construct into the genome of the parent cell at a locus which is not within the first DNA sequence, not within a second DNA sequence encoding a protein that negatively regulates transcription, translation or secretion of the polypeptide, and not within a third DNA sequence encoding a protease which hydrolyzes the polypeptide under the conditions; and the mutant cell produces more of the polypeptide than the parent cell when both cells are cultivated under the conditions; and recovering the polypeptide.

Genzel, Y., et al. describes that substitution of glutamine by pyruvate reduces ammonia formation and growth inhibition of mammalian cells (Biotechnol. Prog. 21 (2005) 58-69). De la cruz Edmonds, M. C., et al. reported the development of transfection and high-producer screening protocols for the CHOK1 SV cell system (Mol. Biotechnol. 34 (2006) 179-190. In WO 2007/036291 an improved cell culture medium is reported. In EP 1 482 031 serum-free mammalian cell culture medium and uses thereof are reported. Link, T., et al. describe about bioprocess development for the production of a recombinant MUC1 fusion protein expressed by CHO-K1 cells in protein-free medium (J. Biotechnol. 110 (2004) 51-62). EP 0 481 791 described a culture medium for CHO-cells and adapted cells. US 2007/161079 describes recombinant cell clones having increased stability and methods of making and using the same. EP 0 659 880 describes a method for culturing animal cells or antibody producing cells. Butler, M., et al. describe the adaptation of mammalian cells to non-ammoniagenic media (Cytotechnol. 15 (1994) 87-94). Altamirano, C. et al., describe improvement of CHO cell culture medium formulation: simultaneous substitution of glucose and glutamine (Biotechnol. Prog. 16 (2000) 69-75).

EP 0 569 678 describes double transfectants of MHC genes as cellular vaccines for immunoprevention of tumor metastasis. WO 97/08342 describes an improved method for measuring the activity of a promoter sequence in a mammalian cell using a reporter gene. The use of anti-RhoA and anti-RhoC siRNAs in order to inhibit specifically RhoA or RhoC synthesis is described in WO 2005/113770. A method for the recombinant production or expression of eukaryotic alkaline phosphatase mutant in yeast cells is described in U.S. Pat. No. 7,202,072. WO 2001/038557 reports a method of screening multiply transformed cells using bicistronic expression of fluorescent proteins. A method for producing recombinant eukaryotic cell lines expressing multiple proteins or RNAs of interest is described in WO 1999/47647. Systems, including methods, compositions, and kits, for transfection of cells with transfection materials using coded carriers are described in WO 2003/076588. U.S. Pat. No. 5,089,397 describes an expression system for recombinant production of a desired protein comprising CHO cells transformed with a DNA sequence having the desired protein coding sequence under the control of the human metallothionein-II promoter. A method for producing recombinant proteins is described in US 2003/0040047. Lamango et al. (Lamango, N. S., et al., Arch. Biochem. Biophys. 330 (1996) 238-250) describe the dependency of the production of prohormone convertase 2 from the presence of the neuroendocrine polypeptide 7B2. The transfection of a BPV-1-based expression vector into cells harboring unintegrated replicating BPV-1 genomes is described by Waldenstroem, M., et al., Gene 120 (1992) 175-181. U.S. Pat. No. 4,912,038 describes methods and vectors for obtaining canine and human 32K alveolar surfactant protein.

WO 89/10959 describes recombinant DNA techniques and the expression of mammalian polypeptides in genetically engineered eukaryotic cells. A repeated co-transfer of an expression vector for human growth hormone and an expression vector for a selection marker gene is described in DD 287531. WO 93/01296 describes antibody production in vaccinia virus infected cells. WO 95/17513 describes retransformation of filamentous fungi. WO 89/00999 describes modular assembly of antibody genes, antibodies prepared thereby and use. US 2003/096341 describes the expression of alkaline phosphatase in yeast.

EP 1 453 966 describes a method for producing a recombinant polypeptide. WO 03/046187 describes a method for producing a recombinant polypeptide. U.S. Pat. No. 5,550,036 describes a method for co-amplification of human protein C genes in human cells. EP 0 921 194 describes a TNF ligand family gene. EP 0 319 206 describes gene amplification. Lin, F. K., et al., describe cloning and expression of the human erythropoietin gene (Proc. Natl. Acad. Sci. USA 82 (1985) 7580-7584). WO 00/28066 describes host cells expressing recombinant human erythropoietin. Chen, S., et al., describe about the production of recombinant proteins in mammalian cells (in Curr. Prot. Prot. Sci. (1998) 5.10.1-5.10.41).

WO 89/00605 describes transfected cells containing vectors having genes oriented in opposing directions and methods of obtaining the same. U.S. Pat. No. 5,420,019 describes stable bactericidal/permeability-increasing protein products and pharmaceutical compositions containing the same. U.S. Pat. No. 5,639,275 describes biocompatible immunoisolatory capsules containing genetically altered cells for the delivery of biologically active molecules. Kemball-Cook, G., et al., describe the high-level production of human blood coagulation factors VII and XI using a new mammalian expression vector (Gene 139 (1994) 275-279). EP 1 010 758 describes an expression system for producing recombinant human erythropoietin, a method for purifying the secreted human erythropoietin and uses thereof.

Mulligan, R. C. and Berg P., describe the selection for animal cells that express the Escherichia coli gene coding for xanthine-guanine phosphoribosyltransferase (Proc. Natl. Acad. Sci. USA 78 (1981) 2072-2076). Colosimo, A., et al., describe the transfer and expression of foreign gene in mammalian cells (BioTechniques 29 (2000) 314-331). Maruyama, K., et al., describe the transfection of cultured mammalian cells by mammalian expression vectors (Meth. Nucleic Acids Res. (1991) 283-305). Wang, D. Z., et al., describe about treating acute stroke patients with intravenous tPA (Stroke 31 (2000) 77-81). Sakamoto, T., et al., describe the prevention of arterial reocclusion after thrombolysis with activated protein C (Circulation 90 (1994) 427-432). Lee, G. M., et al. describe the development of a serum-free medium for the production of erythropoietin by suspension culture of recombinant Chinese hamster ovary cells using a statistical design (J. Biotechnol. 69 (1999) 85-93). Lusky, M. and Botchan, M. R., describe the characterization of the Bovine papilloma virus vector maintenance sequences (Cell 36 (1984) 391-401).

US 2014/0242079 describes a vector ratio of 1:2:1:1 for single expression cassette vectors for the transient expression in HEK cells.

WO 2015/052230 discloses multispecific domain exchanged common variable light chain antibodies.

WO 2012/023053 discloses methods for the generation of multispecific and multivalent antibodies.

WO 2005/072112 discloses methods for producing and identifying multispecific antibodies.

WO 02/079255 discloses recombinant antibodies coexpressed with GnTIII.

US 2002/06210 discloses method for making multispecific antibodies having heteromultimeric and common components.

US 2013/045888 discloses multi-copy strategy for high-titer and high-purity production of multi-subunit proteins such as antibodies in transformed microbes such as Pichia pastoris.

Frenzel et al. reported about the expression of recombinant antibodies in Front. Immunol. 4 (2013) Article 217.

Wurm et al. reported about the production of recombinant protein therapeutics in cultivated mammalian cells (Nat. Biotechnol. 22 (2004) 1393-1398).

### SUMMARY

It has been found that for the generation of cell lines for the production of heterodimeric, i.e. multispecific, antibodies it is advantageous to use an expression vector, which comprises as sole antibody chain expression cassette a light chain expression cassette, for the transfection. This vector can be used together with the other expression vectors in a co-transfection or separately in a second subsequent transfection step. With this approach a production cell line can be obtained that produces the heterodimeric antibody with an improved product profile, i.e. with increased product and reduced product-related impurities.

One aspect as disclosed herein is a method for producing a multispecific antibody, which comprises/is composed of/contains at least three different polypeptides, comprising the following steps:
cultivating a mammalian cell in a cultivation medium (under conditions suitable for the expression of the multispecific antibody), whereby the mammalian cell has been generated by
a) transfecting a mammalian cell (not expressing an antibody) with a first expression vector and one, two or three further expression vectors,
   wherein the first expression vector comprises exactly one nucleic acid sequence encoding a polypeptide of the multispecific antibody, and the one, two or three further expression vectors each comprise at least two nucleic acid sequences each encoding different polypeptide chains of the multispecific antibody,
   wherein the exactly one nucleic acid sequence of the first expression vector is a nucleic acid sequence encoding a light chain polypeptide of the multispecific antibody,
   wherein the transfection with the first expression vector is either concomitant, before or after the transfection with the one, two or three further expression vectors, and
   b) selecting a cell (stably) transfected in step a) growing under selective cultivation conditions,
   recovering the multispecific antibody from the cell or the cultivation medium,

and thereby producing the multispecific antibody.

One aspect as disclosed herein is a method for generating/producing/obtaining a mammalian cell (capable of (stably)) expressing a multispecific antibody, which comprises/is composed of at least three different polypeptides, comprising the following step:
a) transfecting a mammalian cell (not expressing an antibody) with a first expression vector and one, two or three further expression vectors,
   wherein the first expression vector comprises exactly one nucleic acid sequence encoding a polypeptide of the multispecific antibody, and the one, two or three further expression vectors each comprise at least two nucleic acid sequences each encoding different polypeptide chains of the multispecific antibody,
   wherein the exactly one nucleic acid sequence of the first expression vector is a nucleic acid sequence encoding a light chain polypeptide of the multispecific antibody,
   wherein the transfection with the first expression vector is either concomitant, before or after the transfection with the one, two or three further expression vectors, and
b) selecting a cell transfected in step a) growing under selective cultivation conditions,
and thereby generating/producing/obtaining a mammalian cell (stably) expressing a multispecific antibody.

In one embodiment of all aspects as reported herein two of the polypeptides of the multispecific antibody comprise/have a (cognate) domain exchange.

In one embodiment of all aspects as reported herein the exactly one nucleic acid of the first expression vector encodes a light chain polypeptide with a domain exchange of the multispecific antibody.

In one embodiment of all aspects as reported herein step a) comprises: co-transfecting a mammalian cell (not expressing an antibody) with a first expression vector and one, two or three further expression vectors.

In one embodiment of all aspects as reported herein step a) comprises the following steps: i) transfecting (simultaneously or sequentially) a mammalian cell with one, two or three further expression vectors, optionally ii) selecting a (stably) transfected cell, iii) transfecting the cell of i) or ii) with the first expression vector, and optionally iv) selecting a (stably) transfected cell.

In one embodiment of all aspects as reported herein the selecting is based on the expression yield and/or the amount of product-related side-products/impurities.

In one embodiment of all aspects as reported herein the selecting is of the (stably) transfected cell(s) that produce(s) the least amount (fraction) of product-related side-products/impurities.

In one embodiment of all aspects as reported herein the selecting is of the (stably) transfected cell(s) that produce(s) the least amount (fraction) of product-related side-products/impurities and that has the highest yield.

In one embodiment of all aspects as reported herein the mammalian cell stably expresses the multispecific antibody.

In one embodiment of all aspects as reported herein the mammalian cell is a CHO cell.

In one embodiment of all aspects as reported herein the domain exchange is a CH1-CL crossover or a VH-VL-crossover.

In one embodiment of all aspects as reported herein the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

In one embodiment of all aspects as reported herein the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

In one embodiment of all aspects as reported herein the multispecific antibody is a trivalent, bispecific antibody, comprising
a) a first light chain and a first heavy chain of a full length antibody which specifically binds to a first antigen,
b) a second heavy chain of a full length antibody which when paired with the first light chain, specifically binds to the first antigen, and
c) a Fab fragment, which specifically bind to a second antigen, fused via a peptidic linker to the C-terminus of one of the heavy chains of a) or b), wherein the constant domains CL and CH1 of the second light chain and the second heavy chain are replaced by each other.

One aspect as disclosed herein is a (stably transfected) mammalian cell obtained with the method as reported herein.

One aspect as disclosed herein is a method for producing a multispecific antibody comprising the following steps:
cultivating a (stably transfected) cell as disclosed herein in a cultivation medium (under conditions suitable for the expression of the multispecific antibody),
recovering the multispecific antibody from the cell or the cultivation medium,
optionally purifying the recovered antibody with one or more chromatography steps,

and thereby producing the multispecific antibody.

One aspect as disclosed herein is a method for producing a multispecific antibody preparation with low/reduced product-related impurities comprising the following steps:
obtaining/producing/generating a (stably transfected) mammalian cell (stably) expressing a multispecific antibody with a method as disclosed herein,
cultivating the obtained/produced/generated mammalian cell in a cultivation medium,
recovering the antibody preparation from the cell or the cultivation medium,
optionally purifying the recovered antibody with one or more chromatography steps,
and thereby producing a multispecific antibody preparation with low/reduced product-related impurities.

One aspect as disclosed herein is the use of a method as reported herein for reducing product-related impurities in a multispecific antibody preparation.

Herein is reported a method for the production of a multispecific antibody which comprises at least one chain with a domain crossover in a recombinant mammalian cell. The method results in an improved process wherein the improvement is amongst other things a reduction of the product-related side-products and an increase of the amount of correctly folded/correctly assembled multispecific antibody.

One aspect as disclosed herein is a method for producing a multispecific antibody (comprising at least one polypeptide chain with a domain crossover) comprising the following steps:
a) providing a (stably transfected) mammalian cell (stably) expressing the multispecific antibody,
b) transfecting the mammalian cell of step a) with an expression cassette encoding a polypeptide chain of the multispecific antibody that has a domain crossover,
c) cultivating the cell of step b) and recovering the antibody from the cell or the cultivation medium and thereby producing the multispecific antibody,
d) optionally purifying the recovered antibody with one or more chromatography steps.

In one embodiment of all aspects the mammalian cell expressing the multispecific antibody stably expresses the multispecific antibody.

In one embodiment the expression cassette of step b) is in an expression vector.

In one embodiment of all aspects the polypeptide chain of the multispecific antibody that has a domain crossover is an antibody light chain.

In one embodiment of all aspects the domain crossover is a CH1-CL crossover or a VH-VL-crossover.

In one embodiment of all aspects the multispecific antibody is a bivalent bispecific antibody, or a trivalent bispecific antibody, or a tetravalent bispecific antibody.

In one embodiment of all aspects the mammalian cell expressing the multispecific antibody is obtained by transfecting a mammalian cell with one or more nucleic acid molecules encoding the multispecific antibody and selecting a stably transfected cell.

In one embodiment of all aspects the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

In one embodiment of all aspects the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

In one embodiment of all aspects the multispecific antibody is a trispecific or tetraspecific antibody, comprising
a) a first light chain and a first heavy chain of a full length antibody which specifically binds to a first antigen, and
b) a second (modified) light chain and a second (modified) heavy chain of a full length antibody which specifically binds to a second antigen, wherein the variable domains VL and VH are replaced by each other, and/or wherein the constant domains CL and CH1 are replaced by each other, and
c) wherein one to two antigen binding peptides which specifically bind to one or two further antigens (i.e. to a third and/or fourth antigen) are fused via a peptidic linker to the C- or N-terminus of the light chains or heavy chains of a) and/or b).

In one embodiment of all aspects the multispecific antibody is a bi specific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a),
and
wherein in the Fab fragments the following modifications were performed
   i) in both Fab fragments of a), or in both Fab fragments of b), the variable domains VL and VH are replaced by each other, and/or the constant domains CL and CH1 are replaced by each other,
   or
   ii) in both Fab fragments of a) the variable domains VL and VH are replaced by each other, and the constant domains CL and CH1 are replaced by each other,
      and
      in both Fab fragments of b) the variable domains VL and VH are replaced by each other, or the constant domains CL and CH1 are replaced by each other,
   or
   iii) in both Fab fragments of a) the variable domains VL and VH are replaced by each other, or the constant domains CL and CH1 are replaced by each other,
      and
      in both Fab fragments of b) the variable domains VL and VH are replaced by each other, and the constant domains CL and CH1 are replaced by each other,
   or
   iv) in both Fab fragments of a) the variable domains VL and VH are replaced by each other, and in both Fab fragments of b) the constant domains CL and CH1 are replaced by each other,
   or
   v) in both Fab fragments of a) the constant domains CL and CH1 are replaced by each other, and in both Fab fragments of b) the variable domains VL and VH are replaced by each other.

In one embodiment in the Fab fragments the following modifications are performed:
i) in both Fab fragments of a), or in both Fab fragments of b), the variable domains VL and VH are replaced by each other,
   and/or
   the constant domains CL and CH1 are replaced by each other.

In one embodiment of all aspects the multispecific antibody is a bispecific, tetravalent antibody comprising:
a) a (modified) heavy chain of a first antibody, which specifically binds to a first antigen and comprises a first VH-CH1 domain pair, wherein to the C-terminus of said heavy chain is fused to the N-terminus of a second VH-CH1 domain pair of said first antibody via a peptidic linker,
b) two light chains of said first antibody of a),
c) a (modified) heavy chain of a second antibody, which specifically binds to a second antigen and comprises a first VH-CL domain pair, wherein to the C-terminus of said heavy chain is fused to the N-terminus of a second VH-CL domain pair of said second antibody via a peptidic linker, and
d) two (modified) light chains of said second antibody of c), each comprising a CL-CH1 domain pair.

In all aspects as reported herein the first light chain comprises a VL domain and a CL domain and the first heavy chain comprises a VH domain, a CH1 domain, a hinge region, a CH2 domain and a CH3 domain.

In one embodiment of all aspects the antibody as produced in the method as reported herein is a multispecific antibody, which requires heterodimerization of at least two heavy chain polypeptides.

In one embodiment the full length antibody is
a) a full length antibody of the human subclass IgG1,
b) a full length antibody of the human subclass IgG4,
c) a full length antibody of the human subclass IgG1 with the mutations L234A, L235A and P329G,
d) a full length antibody of the human subclass IgG4 with the mutations S228P, L235E and P329G,
e) a full length antibody of the human subclass IgG1 with the mutations L234A, L235A and P329G in both heavy chains and the mutations T366W and S354C or Y349C in one heavy chain and the mutations T366S, L368A, Y407V and Y349C or S354C in the respective other heavy chain,
f) a full length antibody of the human subclass IgG4 with the mutations S228P and P329G in both heavy chains and the mutations T366W and S354C in one heavy chain and the mutations T366S, L368A, Y407V and Y349C in the respective other heavy chain,
g) a full length antibody of the human subclass IgG1 with the mutations L234A, L235A, P329G, I253A, H310A and H435A in both heavy chains and the mutations T366W and S354C in one heavy chain and the mutations T366S, L368A, Y407V and Y349C in the respective other heavy chain, or
h) a full length antibody of the human subclass IgG1 with the mutations L234A, L235A, P329G, M252Y, S254T and T256E in both heavy chains and the mutations T366W and S354C in one heavy chain and the mutations T366S, L368A, Y407V and Y349C in the respective other heavy chain.

One aspect as disclosed herein is a cell comprising a nucleic acid encoding the bispecific antibody obtained with a method as disclosed herein.

One aspect as disclosed herein is a method of producing a multispecific antibody as disclosed herein comprising the following steps:
a) culturing the cell as disclosed herein producing/expressing the multispecific antibody, and
b) recovering the multispecific antibody from the cell or the cultivation medium,

and thereby producing the multispecific antibody as reported herein.

One aspect as disclosed herein is the antibody produced with the method as reported herein.

One aspect as disclosed herein is a pharmaceutical formulation comprising the antibody produced with the method as disclosed herein and a pharmaceutically acceptable carrier.

One aspect as disclosed herein is the antibody produced with the method as disclosed herein for use as a medicament.

One aspect as disclosed herein is the use of the bispecific antibody produced with the method as disclosed herein in the manufacture of a medicament.

In one embodiment of all aspects the bispecific antibody is selected from the group of bispecific antibodies consisting of an anti-Abeta/transferrin receptor antibody, an anti-CD20/transferrin receptor antibody, an anti-PD1/Tim3 antibody, and an anti-FAP/DR5 antibody.

In one embodiment of all aspects the multispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein each of said additional Fab fragments is fused via a peptidic linker to an individual C-terminus of one of the heavy chains of a),
and
wherein in the additional Fab fragments the following modifications were performed
   in both additional Fab fragments of b), the variable domains VL and VH are replaced by each other, and/or the constant domains CL and CH1 are replaced by each other,
wherein i) the first antigen is DR5 and the second antigen is FAP, or ii) the first antigen is FAP and the second antigen is DR5,
wherein the two heavy chains of an antibody, which specifically bind to a first antigen are of the human subclass IgG1 with the mutations L234A, L235A and P329G.

In one embodiment of all aspects the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.
wherein i) the first antigen is PD1 and the second antigen is Tim3, or ii) the first antigen is Tim3 and the second antigen is PD1,
wherein the first heavy chain and the second heavy are both of the human subclass IgG1 with the mutations L234A, L235A and P329G and with the mutation T366W and optionally S354C or Y349C in one heavy chain and the mutations T366S, L368A, Y407V and optionally Y349C or S354C in the respective other heavy chain, whereby the terminal glycine or glycine-lysine dipeptide can be absent,
wherein the first light chain comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein the first heavy chain comprises in the first constant heavy chain domain (CH1) at position 147 a glutamic acid residue (instead of the wild-type lysine residue; K147E mutation) and at position 213 a glutamic acid residue (instead of the wild-type lysine amino acid residue; K213E mutation) (numbering according to Kabat EU index).

In one embodiment of all aspects the multispecific antibody is a trivalent, bispecific antibody comprising
a) two light chains and two heavy chains of an antibody, which specifically bind to a first antigen (and comprise two Fab fragments),
b) one additional Fab fragment of an antibody, which specifically bind to a second antigen, wherein said additional Fab fragment is fused via a peptidic linker to the C-terminus of one of the heavy chains of a),
and
wherein in the additional Fab fragment the following modifications were performed
   the variable domains VL and VH are replaced by each other, and/or the constant domains CL and CH1 are replaced by each other,
wherein i) the first antigen is Abeta and the second antigen is the transferrin receptor, or ii) the first antigen is CD20 and the second antigen is the transferrin receptor.

In one embodiment of all aspects the multispecific antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 a glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other, and
wherein the first antigen is human A-beta protein and the second antigen is human transferrin receptor.

In one embodiment of all aspects the multispecific antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 a glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other, and
wherein the first antigen is human CD20 and the second antigen is human transferrin receptor.

In one embodiment of all aspects as reported herein each polypeptide is within an expression cassette each comprising in 5′- to 3′-direction a promoter, a structural gene encoding the polypeptide, a polyadenylation sequence and optionally a terminator sequence. In one embodiment all expression cassettes have the same promoter, the same polyadenylation site and optionally the same terminator sequence. In one embodiment the promoter is the human CMV (cytomegalovirus) promoter. In one embodiment the CMV promoter comprises an intron A. In one embodiment the polyadenylation site is the BGH (bovine growth hormone) polyadenylation site. In one embodiment the terminator is present and is the HGT (human growth hormone terminator). In one embodiment the promoter is the CMV promoter optionally comprising an intron A and the polyadenylation site is the BGH polyadenylation site. In one embodiment the promoter is the CMV promoter optionally comprising an intron A, the polyadenylation site is the BGH polyadenylation site and the terminator is the HGT.

In one embodiment the further expression vector comprises or each of the further expression vectors each comprises at least two nucleic acid sequences each encoding different polypeptide chains of the multispecific antibody, wherein each encoding nucleic acid is present/contained exactly once on the respective vector.

### DETAILED DESCRIPTION OF THE INVENTION

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

### Definitions

A “multispecific antibody” denotes an antibody that has binding specificities for at least two different epitopes on the same antigen or two different antigens. Multispecific antibodies can be prepared as full-length antibodies or antibody fragments (e.g. F(ab′)2 bispecific antibodies) or combinations thereof (e.g. full length antibody plus additional scFv or Fab fragments). Engineered antibodies with two, three or more (e.g. four) functional antigen binding sites have also been reported (see, e.g., US 2002/0004587 A1).

The term “correctly folded/correctly assembled” as used herein denotes that the antibody has the correct stoichiometry, i.e. comprises the matching number and copies of the individual/respective light and heavy chains. For example, a “native human IgG antibody” is correctly folded/correctly assembled when an isolated molecule comprises two light chain polypeptides and two heavy chain polypeptides. For example, if the multispecific antibody is a bivalent, bispecific native human IgG antibody which is correctly folded/correctly assembled when the isolated molecule is consisting of a first pair of a cognate first light chain and a cognate first heavy chain binding to a first antigen and a second pair of a cognate second light chain and a cognate second heavy chain binding to a second antigen, i.e. of four different polypeptides. All antibodies that are not correctly folded/correctly assembled, i.e. that comprise less or more than the required number of chains and/or comprise wrongly associated chains, i.e. not forming a cognate pair of a heavy and light chain, are termed “product-related side-products”.

The term “domain crossover” as used herein denotes that in a pair of an antibody heavy chain VH-CH1 fragment and its corresponding cognate antibody light chain, i.e. in an antibody binding arm (i.e. in the Fab fragment), the domain sequence deviates from the natural sequence in that at least one heavy chain domain is substituted by its corresponding light chain domain and vice versa. There are three general types of domain crossovers, (i) the crossover of the CH1 and the CL domains, which leads to domain crossover light chain with a VL-CH1 domain sequence and a domain crossover heavy chain fragment with a VH-CL domain sequence (or a full length antibody heavy chain with a VH-CL-hinge-CH2-CH3 domain sequence), (ii) the domain crossover of the VH and the VL domains, which leads to domain crossover light chain with a VH-CL domain sequence and a domain crossover heavy chain fragment with a VL-CH1 domain sequence, and (iii) the domain crossover of the complete light chain (VL-CL) and the complete VH-CH1 heavy chain fragment (“Fab crossover”), which leads to a domain crossover light chain with a VH-CH1 domain sequence and a domain crossover heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH1 and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W. et al, Proc. Natl. Acad. Sci USA 108 (2011) 11187-11192.

The multispecific antibody produced with a method as reported herein essentially comprises Fab fragments including a domain crossover of the CH1 and the CL domains as mentioned under item (i) above, or a domain crossover of the VH and the VL domains as mentioned under item (ii) above. The Fab fragments specifically binding to the same antigen(s) are constructed to be of the same domain sequence. Hence, in case more than one Fab fragment with a domain crossover is contained in the multispecific antibody, said Fab fragment(s) specifically bind to the same antigen.

The term “antibody” herein is used in the broadest sense and encompasses various antibody structures, including but not limited to monoclonal antibodies, polyclonal antibodies, and multispecific antibodies (e.g., bispecific antibodies) so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc receptor” as used herein refers to activation receptors characterized by the presence of a cytoplasmatic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA The term “no binding of FcγR” denotes that at an antibody concentration of 10 μg/ml the binding of an antibody as produced in the method as reported herein to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434). In one embodiment the antibody as produced in the method as reported herein is of IgG1 or IgG2 subclass and comprises the mutation PVA236, GLPSS331, and/or L234A/L235A. In one embodiment the antibody as produced in the method as reported herein is of IgG4 subclass and comprises the mutation L235E. In one embodiment the antibody further comprises the mutation S228P.

The term “Fe-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242.

The antibodies as produced in the method as reported herein comprise as Fc-region, in one embodiment an Fc-region derived from human origin. In one embodiment the Fc-region comprises all parts of the human constant region. The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat; Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies. In one embodiment the Fc-region is a human Fc-region. In one embodiment the Fc-region is of the human IgG4 subclass comprising the mutations S228P and/or L235E (numbering according to EU index of Kabat). In one embodiment the Fc-region is of the human IgG1 subclass comprising the mutations L234A and L235A (numbering according to EU index of Kabat).

The terms “full length antibody”, “intact antibody,” and “whole antibody” are used herein interchangeably to refer to an antibody having a structure substantially similar to a native antibody structure or having heavy chains that contain an Fc-region as defined herein. A “full length antibody” is an antibody that comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. In more detail a full length antibody comprises two antibody light chains (each comprising a light chain variable domain and a light chain constant domain) and two antibody heavy chains (each comprising a heavy chain variable domain, a hinge region and the heavy chain constant domains CH1, CH2 and CH3). The C-terminal amino acid residues K or GK may be present or not independently of each other in the two antibody heavy chains of a full length antibody.

The terms “cell”, “cell line”, and “cell culture” are used interchangeably and refer to cells into which exogenous nucleic acid has been introduced, including the progeny of such cells. Cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), whereby between the first and the second constant domain a hinge region is located. Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain. A “native-like” antibody has the same structure as a “native antibody” but a different binding specificity.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “expression cassette” denotes a construct that contains the necessary regulatory elements, such as promoter and polyadenylation site, for expression of at least the contained nucleic acid in a cell.

The term “expression vector” denotes a nucleic acid providing all required elements for the expression of the comprised structural gene(s) in a cell. Typically, an expression vector comprises a prokaryotic plasmid propagation unit, e.g. for E. coli, comprising an origin of replication, and a selection marker, an eukaryotic selection marker, and one or more expression cassettes for the expression of the structural gene(s) of interest each comprising a promoter nucleic acid, a structural gene, and a transcription terminator including a polyadenylation signal. Gene expression is usually placed under the control of a promoter nucleic acid, and such a structural gene is said to be “operably linked to” the promoter nucleic acid. Similarly, a regulatory element and a core promoter nucleic acid are operably linked if the regulatory element modulates the activity of the core promoter nucleic acid.

The term “operably linked” denotes a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader and a polypeptide, contiguous and in (reading) frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distance from the promoter. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

The term “polypeptide” denotes a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “producing” denotes the expression of a structural gene inserted into an expression cassette in a cell. The term includes the processes of transcription and translation of nucleic acid. Producing is performed in appropriate prokaryotic or eukaryotic cells and the expressed, i.e. produced, polypeptide can be recovered from the cells after lysis or from the culture supernatant.

The term “promoter nucleic acid” denotes a polynucleotide sequence that controls transcription of a gene/structural gene or nucleic acid sequence to which it is operably linked. A promoter nucleic acid includes signals for RNA polymerase binding and transcription initiation. The used promoter nucleic acid will be functional in the cell in which expression of the selected structural gene is contemplated. A large number of promoter nucleic acids including constitutive, inducible and repressible promoters from a variety of different sources are well known in the art (and identified in databases such as GenBank) and are available as or within cloned polynucleotides (from, e.g., depositories such as ATCC as well as other commercial or individual sources).

Typically, a promoter nucleic acid is located in the 5′ non-coding or untranslated region of a gene, proximal to the transcriptional start site of the structural gene. Sequence elements within promoter nucleic acids that function in the initiation of transcription are often characterized by consensus nucleotide sequences. These elements include RNA polymerase binding sites, TATA sequences, CAAT sequences, differentiation-specific elements (DSEs), cyclic AMP response elements (CREs), serum response elements (SREs), glucocorticoid response elements (GREs), and binding sites for other transcription factors, such as CRE/ATF, AP2, SP1, cAMP response element binding protein (CREB) and octamer factors. If a promoter nucleic acid is an inducible promoter nucleic acid, then the rate of transcription increases in response to an inducing agent, such as a CMV promoter nucleic acid followed by two tet-operator site, the metallothionein and heat shock promoter nucleic acids. The rate of transcription is not regulated by an inducing agent if the promoter nucleic acid is a constitutively active promoter nucleic acid. Among the eukaryotic promoter nucleic acids that have been identified as strong promoter nucleic acids for expression are the SV40 early promoter nucleic acid, the adenovirus major late promoter nucleic acid, the mouse metallothionein-I promoter nucleic acid, the Rous sarcoma virus long terminal repeat, the Chinese hamster elongation factor 1 alpha (CHEF-1), human EF-1 alpha, ubiquitin, and human cytomegalovirus major-immediate-early promoter nucleic acid (hCMV MIE).

The term “selection marker” denotes a nucleic acid that allows cells carrying it to be specifically selected for or against, in the presence of a corresponding selection agent (cultivation under selective cultivation conditions). Typically, a selection marker will confer resistance to a drug or compensate for a metabolic or catabolic defect in the cell into which it is introduced. A selection marker can be positive, negative, or bifunctional. A useful positive selection marker is an antibiotic resistance gene allowing for the selection of cells transformed therewith in the presence of the corresponding selection agent, e.g. the antibiotic. A non-transformed cell is not capable to grow or survive under the selective conditions, i.e. in the presence of the selection agent. Negative selection markers allow cells carrying the marker to be selectively eliminated. Selection markers used with eukaryotic cells include, e.g., the structural genes encoding aminoglycoside phosphotransferase (APH), such as e.g. the hygromycin (hyg), neomycin (neo), and G418 selection markers, dihydrofolate reductase (DHFR), thymidine kinase (tk), glutamine synthetase (GS), asparagine synthetase, tryptophan synthetase (selection agent indole), histidinol dehydrogenase (selection agent histidinol D), and nucleic acids conferring resistance to puromycin, bleomycin, phleomycin, chloramphenicol, Zeocin, and mycophenolic acid.

### Methods

The current invention is based at least in part on the finding that for the generation of cell lines for the production of heterodimeric antibodies it is advantageous to use in the transfection an expression vector which comprises as sole (antibody) polypeptide encoding nucleic acid a light chain polypeptide encoding nucleic acid, i.e. the vector comprises as sole antibody polypeptide expression cassette a light chain expression cassette. This vector is used together with further expression vectors in a co-transfection or separately in a second subsequent transfection step. With this approach a production cell line can be obtained that produces the heterodimeric antibody with an improved product profile, i.e. with increased product and reduced product-related impurities.

One approach for designing multispecific antibodies is known as the “CrossMab technology”. This approach is based on a domain crossover between heavy and light chains thereby creating different domain arrangements for heavy chains and light chains of different specificity (see e.g. WO 2009/080251, WO 2009/080252, WO 2009/080253, WO 2009/080254, Schaefer, W. et al. Proc. Natl. Acad. Sci. USA 108 (2011) 11187-11192 relating to bivalent, bispecific IgG antibodies with a domain crossover; WO 2010/145792 and WO 2010/145792 relate to tetravalent antigen binding proteins with a domain crossover).

The multispecific antibodies with a VH/VL replacement/exchange in one binding site to prevent light chain mispairing (CrossMabVH-VL) which are described in WO2009/080252, (see also Schaefer, W. et al, PNAS, 108 (2011) 11187-1191) clearly reduce the byproducts caused by the mismatch of a light chain against a first antigen with the wrong heavy chain against the second antigen (compared to approaches without such domain exchange). However, their preparation is not completely free of side products. The main side product is based on a Bence-Jones-type interaction of the wrong light chain with the domain-exchanged heavy chain (see also Schaefer, W. et al, PNAS, 108 (2011) 11187-1191; in Fig. S1I of the Supplement).

WO2015/101588 A1 relates to blood brain barrier shuttle modules. WO2015/101588 A1 mentions bivalent, bispecific antibodies with a VH/VL domain crossover in one of the binding arms with mutations in the CH1/CL interface. WO 2015/101588 A1 is silent on the technical effect of said mutations.

Various methods for generating cell lines for producing four-chain homodimeric bivalent antibodies, i.e. native-like antibodies, are known. To increase the productivity of such cells lines some of these methods rely on a so-called “supertransfection” approach. Therein the cells are transfected at least two-times with intermediate cell line selection. The vectors used in this supertransfection approach each normally comprise the entire coding information for the antibody to be expressed, i.e. for the light chain and for the heavy chain. Some special supertransfection methods employ very similar or even identical vectors differing only in the selection marker in order to achieve close-by integration into the genome in a known productive region. Like the gene amplification methods using DHFR the supertransfection methods aim at increasing the expression yield by increasing the number of functional expression cassettes in the cells.

For novel complex trivalent, bispecific antibody formats comprising a heterodimeric Fc-region and a so-called domain exchange, which both are introduced in order to limit or even exclude chain mispairing and thereby increase the yield of correctly folded and assembled multispecific antibody obtained, a complex procedure of co-transfection of three to four vectors each comprising a single expression cassette at different vector ratios, has been reported (see e.g. WO 2013/026833).

The invention is based, at least in part, on the finding that the expression yield of a multispecific antibody of a recombinant cell can be improved if the cell is re-transfected with an expression cassette for the expression of the light chain of said multispecific antibody. This is especially useful if the multispecific antibody comprises variant heavy and light chains with domain crossover.

One aspect as disclosed herein is a method for producing a multispecific antibody (comprising at least one polypeptide with a domain crossover) comprising the following steps:
a) providing a mammalian cell expressing the antibody,
b) transfecting the mammalian cell of a) with an expression vector comprising an expression cassette encoding a polypeptide of the antibody that has a domain crossover,
c) cultivating the cell of b) and recovering the antibody from the cell or the cultivation medium and thereby producing the multispecific antibody.

The modified cell obtained with the method as reported herein “secrets” more of the multispecific antibody in correctly folded and assembled form and is defined herein as a cell in which the amount of the correctly folded and correctly assembled multispecific antibody released into the extracellular medium is increased relative to the parent cell. Immunoblot analysis, biological activity assays, and physical-chemical separation methods may be used to quantify the absolute amounts of the correctly folded and assembled multispecific antibody released by the modified cell vs. the parent cell.

One aspect as disclosed herein is a method of producing a multispecific antibody comprising the following steps:
a) cultivating a modified cell under conditions suitable/conducive for production of the multispecific antibody, wherein
   i) the modified cell is related to a parent cell, wherein the parent cell comprises first DNA sequences encoding the multispecific antibody, by the introduction of a nucleic acid into the genome of the parent cell at a locus which is not within the first DNA sequences; and
   ii) the modified cell produces more of the multispecific antibody than the parent cell when both cells are cultivated under the same conditions; and
b) recovering the polypeptide.
   Antibody Formats with Domain Crossover

The method as reported herein is generally suitable for the production of any multispecific antibody comprising separately encoded heavy and light chain.

In one embodiment the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

The antibody under a) does not contain a modification as reported under b) and the heavy chain and the light chain under a) are isolated chains.

In the antibody under b)

within the light chain
the variable light chain domain VL is replaced by the variable heavy chain domain VH of said antibody,

and

within the heavy chain
the variable heavy chain domain VH is replaced by the variable light chain domain VL of said antibody.

In one embodiment the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other.

The antibody under a) does not contain a modification as reported under b) and the heavy chain and the light chain and a) are isolated chains.

In the antibody under b)

within the light chain
the variable light chain domain VL is replaced by the variable heavy chain domain VH of said antibody, and the constant light chain domain CL is replaced by the constant heavy chain domain CH1 of said antibody;

and

within the heavy chain
the variable heavy chain domain VH is replaced by the variable light chain domain VL of said antibody, and the constant heavy chain domain CH1 is replaced by the constant light chain domain CL of said antibody.

In one embodiment the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

The antibody under a) does not contain a modification as reported under b) and the heavy chain and the light chain under a) are isolated chains.

In the antibody under b)

within the light chain
the constant light chain domain CL is replaced by the constant heavy chain domain CH1 of said antibody;

and within the heavy chain
the constant heavy chain domain CH1 is replaced by the constant light chain domain CL of said antibody.

In one embodiment the multispecific antibody is a trispecific or tetraspecific antibody, comprising
a) a first light chain and a first heavy chain of a full length antibody which specifically binds to a first antigen, and
b) a second (modified) light chain and a second (modified) heavy chain of a full length antibody which specifically binds to a second antigen, wherein the variable domains VL and VH are replaced by each other, and/or wherein the constant domains CL and CH1 are replaced by each other, and
c) wherein one to four antigen binding peptides which specifically bind to one or two further antigens (i.e. to a third and/or fourth antigen) are fused via a peptidic linker to the C- or N-terminus of the light chains or heavy chains of a) and/or b).

The antibody under a) does not contain a modification as reported under b) and the heavy chain and the light chain and a) are isolated chains.

In one embodiment the trispecific or tetraspecific antibody comprises under c) one or two antigen binding peptides which specifically bind to one or two further antigens.

In one embodiment the antigen binding peptides are selected from the group of a scFv fragment and a scFab fragment.

In one embodiment the antigen binding peptides are scFv fragments.

In one embodiment the antigen binding peptides are scFab fragments.

In one embodiment the antigen binding peptides are fused to the C-terminus of the heavy chains of a) and/or b).

In one embodiment the trispecific or tetraspecific antibody comprises under c) one or two antigen binding peptides which specifically bind to one further antigen.

In one embodiment the trispecific or tetraspecific antibody comprises under c) two identical antigen binding peptides which specifically bind to a third antigen. In one preferred embodiment such two identical antigen binding peptides are fused both via the same peptidic linker to the C-terminus of the heavy chains of a) and b). In one preferred embodiment the two identical antigen binding peptides are either a scFv fragment or a scFab fragment.

In one embodiment the trispecific or tetraspecific antibody comprises under c) two antigen binding peptides which specifically bind to a third and a fourth antigen. In one embodiment said two antigen binding peptides are fused both via the same peptide connector to the C-terminus of the heavy chains of a) and b). In one preferred embodiment said two antigen binding peptides are either a scFv fragment or a scFab fragment.

In one embodiment the multispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a),
and
wherein in the Fab fragments the following modifications were performed
   i) in both Fab fragments of a), or in both Fab fragments of b), the variable domains VL and VH are replaced by each other, and/or the constant domains CL and CH1 are replaced by each other,
   or
   ii) in both Fab fragments of a) the variable domains VL and VH are replaced by each other, and the constant domains CL and CH1 are replaced by each other,
      and
      in both Fab fragments of b) the variable domains VL and VH are replaced by each other, or the constant domains CL and CH1 are replaced by each other,
   or
   iii) in both Fab fragments of a) the variable domains VL and VH are replaced by each other, or the constant domains CL and CH1 are replaced by each other,
      and
      in both Fab fragments of b) the variable domains VL and VH are replaced by each other, and the constant domains CL and CH1 are replaced by each other,
   or
   iv) in both Fab fragments of a) the variable domains VL and VH are replaced by each other, and in both Fab fragments of b) the constant domains CL and CH1 are replaced by each other,
   or
   v) in both Fab fragments of a) the constant domains CL and CH1 are replaced by each other, and in both Fab fragments of b) the variable domains VL and VH are replaced by each other.

In one embodiment said additional Fab fragments are fused both via a peptidic linker either to the C-termini of the heavy chains of a), or to the N-termini of the heavy chains of a).

In one embodiment said additional Fab fragments are fused both via a peptidic linker either to the C-termini of the heavy chains of a).

In one embodiment said additional Fab fragments are fused both via a peptide connector to the N-termini of the heavy chains of a).

In one embodiment in the Fab fragments the following modifications are performed:
i) in both Fab fragments of a), or in both Fab fragments of b), the variable domains VL and VH are replaced by each other,
   and/or
   the constant domains CL and CH1 are replaced by each other.

In one embodiment in the Fab fragments the following modifications are performed:
i) in both Fab fragments of a) the variable domains VL and VH are replaced by each other,
   and/or
   the constant domains CL and CH1 are replaced by each other.

In one embodiment in the Fab fragments the following modifications are performed:
i) in both Fab fragments of a) the constant domains CL and CH1 are replaced by each other.

In one embodiment in the Fab fragments the following modifications are performed:
i) in both Fab fragments of b) the variable domains VL and VH are replaced by each other,
   and/or
   the constant domains CL and CH1 are replaced by each other.

In one embodiment in the Fab fragments the following modifications are performed:
i) in both Fab fragments of b) the constant domains CL and CH1 are replaced by each other.

In one embodiment the multispecific antibody is a bispecific, tetravalent antibody comprising:
a) a (modified) heavy chain of a first antibody, which specifically binds to a first antigen and comprises a first VH-CH1 domain pair, wherein to the C-terminus of said heavy chain the N-terminus of a second VH-CH1 domain pair of said first antibody is fused via a peptidic linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptidic linker, and
d) two (modified) light chains of said second antibody of c), each comprising a CL-CH1 domain pair.

In one embodiment the multispecific antibody is a bispecific antibody comprising
a) the heavy chain and the light chain of a first full length antibody that specifically binds to a first antigen, and
b) the heavy chain and the light chain of a second full length antibody that specifically binds to a second antigen, wherein the N-terminus of the heavy chain is connected to the C-terminus of the light chain via a peptidic linker.

The antibody under a) does not contain a modification as reported under b) and the heavy chain and the light chain are isolated chains.

In all aspects as reported herein the first light chain comprises a VL domain and a CL domain and the first heavy chain comprises a VH domain, a CH1 domain, a hinge region, a CH2 domain and a CH3 domain.

In one embodiment the antibody as produced in the method as reported herein is a multispecific antibody, which requires heterodimerization of at least two heavy chain polypeptides.

Several approaches for CH3-modifications in order to support heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, which are herein included by reference. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CH3-engineered first heavy chain can no longer homodimerize with another CH3-engineered first heavy chain; and a CH3-engineered second heavy chain can no longer homodimerize with another CH3-engineered second heavy chain). Thereby the heavy chain comprising one engineered CH3 domain is forced to heterodimerize with another heavy chain comprising the CH3 domain, which is engineered in a complementary manner. For this embodiment of the invention, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g. for steric reasons).

The different approaches for supporting heavy chain heterodimerization known in the art, that were cited and included above, are contemplated as different alternatives used in a multispecific antibody according to the invention, which comprises a “non-crossed Fab region” derived from a first antibody, which specifically binds to a first antigen, and a “crossed Fab region” derived from a second antibody, which specifically binds to a second antigen, in combination with the particular amino acid substitutions described above for the invention.

The CH3 domains of the multispecific antibody as produced in the method as reported herein can be altered by the “knob-into-holes” technology which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

In one preferred embodiment the multispecific antibody as produced in the method as reported herein comprises a T366W mutation in the CH3 domain of the “knobs chain” and T366S, L368A, Y407V mutations in the CH3 domain of the “hole-chain” (numbering according to Kabat EU index). An additional interchain disulfide bridge between the CH3 domains can also be used (Merchant, A. M., et al., Nature Biotech. 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the multispecific antibody as produced in the method as reported herein comprises the Y349C and T366W mutations in one of the two CH3 domains and the E356C, T366S, L368A and Y407V mutations in the other of the two CH3 domains or the multispecific antibody as produced in the method as reported herein comprises the Y349C and T366W mutations in one of the two CH3 domains and the S354C, T366S, L368A and Y407V mutations in the other of the two CH3 domains (the additional Y349C mutation in one CH3 domain and the additional E356C or S354C mutation in the other CH3 domain forming a interchain disulfide bridge) (numbering according to Kabat EU index).

But also other knobs-in-holes technologies as described by EP 1 870 459A1, can be used alternatively or additionally. In one embodiment the multispecific antibody as produced in the method as reported herein comprises the R409D and K370E mutations in the CH3 domain of the “knobs chain” and the D399K and E357K mutations in the CH3 domain of the “hole-chain” (numbering according to Kabat EU index).

In one embodiment the multispecific antibody as produced in the method as reported herein comprises a T366W mutation in the CH3 domain of the “knobs chain” and the T366S, L368A and Y407V mutations in the CH3 domain of the “hole chain” and additionally the R409D and K370E mutations in the CH3 domain of the “knobs chain” and the D399K and E357K mutations in the CH3 domain of the “hole chain” (numbering according to the Kabat EU index).

In one embodiment the multispecific antibody as produced in the method as reported herein comprises the Y349C and T366W mutations in one of the two CH3 domains and the S354C, T366S, L368A and Y407V mutations in the other of the two CH3 domains, or the multispecific antibody as produced in the method as reported herein comprises the Y349C and T366W mutations in one of the two CH3 domains and the S354C, T366S, L368A and Y407V mutations in the other of the two CH3 domains and additionally the R409D and K370E mutations in the CH3 domain of the “knobs chain” and the D399K and E357K mutations in the CH3 domain of the “hole chain” (numbering according to the Kabat EU index).

Apart from the “knob-into-hole technology” other techniques for modifying the CH3 domains of the heavy chains of a multispecific antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with a multispecific antibody as produced in the method as reported herein.

In one embodiment of all aspects and embodiments as reported herein the multispecific antibody is a bispecific antibody or a trispecific antibody. In one preferred embodiment of the invention the multispecific antibody is a bispecific antibody.

In one embodiment of all aspects as reported herein, the antibody is a bivalent or trivalent antibody. In one embodiment the antibody is a bivalent antibody.

In one embodiment of all aspects as reported herein, the multispecific antibody has a constant domain structure of an IgG type antibody. In one further embodiment of all aspects as reported herein, the multispecific antibody is characterized in that said multispecific antibody is of human subclass IgG1, or of human subclass IgG1 with the mutations L234A and L235A. In one further embodiment of all aspects as reported herein, the multispecific antibody is characterized in that said multispecific antibody is of human subclass IgG2. In one further embodiment of all aspects as reported herein, the multispecific antibody is characterized in that said multispecific antibody is of human subclass IgG3. In one further embodiment of all aspects as reported herein, the multispecific antibody is characterized in that said multispecific antibody is of human subclass IgG4 or, of human subclass IgG4 with the additional mutation S228P. In one further embodiment of all aspects as reported herein, the multispecific antibody is characterized in that said multispecific antibody is of human subclass IgG1 or human subclass IgG4. In one further embodiment of all aspects as reported herein, the multispecific antibody is characterized in that said multispecific antibody is of human subclass IgG1 with the mutations L234A and L235A (numbering according to Kabat EU index). In one further embodiment of all aspects as reported herein, the multispecific antibody is characterized in that said multispecific antibody is of human subclass IgG1 with the mutations L234A, L235A and P329G (numbering according to Kabat EU index). In one further embodiment of all aspects as reported herein, the multispecific antibody is characterized in that said multispecific antibody is of human subclass IgG4 with the mutations S228P and L235E (numbering according to Kabat EU index). In one further embodiment of all aspects as reported herein, the multispecific antibody is characterized in that said multispecific antibody is of human subclass IgG4 with the mutations S228P, L235E and P329G (numbering according to Kabat EU index).

In one embodiment of all aspects as reported herein, an antibody comprising a heavy chain including a CH3 domain as specified herein, comprises an additional C-terminal glycine-lysine dipeptide (G446 and K447, numbering according to Kabat EU index). In one embodiment of all aspects as reported herein, an antibody comprising a heavy chain including a CH3 domain, as specified herein, comprises an additional C-terminal glycine residue (G446, numbering according to Kabat EU index).

### Bispecific, Trivalent Anti-Human A-Beta/Human Transferrin Receptor Antibody

This antibody is a bispecific antibody consisting of a full-length core antibody and a fused Fab fragment in which certain domains are crosswise exchanged. Thus, the resulting bispecific antibody is asymmetric. Therefore, the bispecific antibody is produced using the heterodimerization technology called knobs-into-holes using a first heavy chain with the so-called knob mutations (HCknob) and a second heavy chain with the so-called hole mutations (HChole).

In this example a co-transfection has been used.

Antibody 0012, antibody 0015, antibody 0020 and antibody 0024 are reported in WO 2017/055540 A1 (SEQ ID NO: 06 to 09, SEQ ID NO: 01 to 03 and SEQ ID NO: 10, SEQ ID NO: 11 to 13 and SEQ ID NO: 14 to 17 of WO 2017/055540 A1, respectively).

Antibody 0012 is a full length antibody comprising one heavy chain with the hole mutations and one heavy chain with the knob mutations, wherein to the C-terminus of the heavy chain with the knob mutations the VL of a Fab is fused via a linker, wherein in the Fab the VH and VL domains are exchanged (VH-VL domain crossover). Both Fabs without domain crossover of the full length antibody have been modified to comprise charges to assist correct assembly.

Antibody 0015 is a full length antibody comprising one heavy chain with the hole mutations and one heavy chain with the knob mutations, wherein to the C-terminus of the heavy chain with the knob mutations the VH of a Fab is fused via a linker, wherein in the Fab the CH1 and CL domains are exchanged (CH-CL domain crossover). Both Fabs without domain crossover of the full length antibody have been modified to comprise charges to assist correct assembly.

Antibody 0020 is a full length antibody comprising one heavy chain with the hole mutations and one heavy chain with the knob mutations, wherein to the C-terminus of the heavy chain with the knob mutations the VL of a single chain Fab is fused via a linker (no domain crossover). Both Fabs without domain crossover have been modified to comprise charges to assist correct assembly.

Antibody 0024 is a full length antibody comprising one heavy chain with the hole mutations and one heavy chain with the knob mutations, wherein to the C-terminus of the heavy chain with the knob mutations the VH of a Fab is fused via a linker, wherein in the Fab the CH1 and CL domains are exchanged (CH-CL domain crossover).

Different allocation/combination of the respective polypeptides on different expression vectors, different ratios of the resulting vectors and different transfection sequences have been used for the recombinant production of the bispecific antibodies.

LC+HChole: expression vector comprising one expression cassette for the heavy chain with the hole mutation and the light chain.

LCcross+HCknob: expression vector comprising one expression cassette for the heavy chain with the knob mutation and the light chain with domain crossover.

LC: expression vector comprising one expression cassette for the light chain.

LCcross: expression vector comprising one expression cassette for the light chain with domain crossover.

HCknob: expression vector comprising one expression cassette for the heavy chain with knob mutation and a fused scFab.

The results in CHO-K1 cells are presented in the following Table.

| | | relative peak area [%] | | | |
|---|---|---|---|---|---|
| | | (non-reduced analysis) | | | |
| | | | | hole- | |
| | | | | hole | |
| | | LC or | ½ mAb | chain | antibody |
| antibody | vector ratio | LCcross | hole | dimer | monomer |
| 0012 | 1(LC + | | 12 | 9 | 78 |
| | HChole):3(LCcross + | | | | |
| | HCknob) | | | | |
| 0012 | 1(LC):1(LC + | 1 | 9 | 9 | 79 |
| | HChole):3(LCcross + | | | | |
| | HCknob) | | | | |
| 0012 | 1(LC + | 6 | 9 | 9 | 75 |
| | HChole):3(LCcross + | | | | |
| | HCknob):1(LCcross) | | | | |
| 0015 | 1(LC + | | 7 | 23 | 62 |
| | HChole):3(LCcross + | | | | |
| | HCknob) | | | | |
| 0015 | 1(LC):1(LC + | | 4 | 17 | 75 |
| | HChole):3(LCcross + | | | | |
| | HCknob) | | | | |
| 0015 | 1(LC + | | 4 | 20 | 66 |
| | HChole):3(LCcross + | | | | |
| | HCknob):1(LCcross) | | | | |
| 0020 | 1(LC + | | 16 | 11 | 72 |
| | HChole):4(HCknob) | | | | |

The bispecific antibodies have been produced in small scale in CHO-S cells and the by-product distribution has been analyzed after a first purification step using a protein A affinity chromatography and after the second purification step using a preparative size-exclusion chromatography. The results are presented in the following Table.

| | | | by-product distribution | | |
|---|---|---|---|---|---|
| | | harvest 3-liter | (CE-SDS not red.) | | |
| | | fermentation after | | | hole- |
| | | preparative protein | | | hole |
| | | A product monomer | | ½ | dimer + |
| | vector | (CE-SDS not red./ | LC or | mAb | ½ mAb |
| antibody | ratio | yield) | LCcross | hole | knob |
| 0012 | 1:1:3 | 65% | 3% | 28% | 3.5% |
| | | 13.3 mg | | | |
| 0024 | 1:1:3 | 70% | 6% | 15% | 7% |
| | | 14.8 mg | | | |
| 0015 | 1:1:3 | 85% | 4% | 5% | 5% |
| | | 15.8 mg | | | |
| 0020 | 1:4 | 29% | 11% | 44% | 8% |
| | | 6 mg | | | |

| | | harvest 3-liter | | | |
|---|---|---|---|---|---|
| | | fermentation after | by-product distribution | | |
| | | preparative protein | (CE-SDS not red.) | | |
| | | A and preparative | | | hole- |
| | | SEC product monomer | | ½ | hole |
| | plasmid | (CE-SDS not red./ | | mAb | dimer + |
| antibody | ratio | yield) | LC | hole | ¾ mAb |
| 0012 | 1:1:3 | >90% | 5% | 3% | 2.5% |
| | | 2.8 mg | | | |
| 0024 | 1:1:3 | 78% | 11% | 5% | 6% |
| | | 4 mg | | | |
| 0015 | 1:1:3 | >95% | 1% | 0.5% | 1% |
| | | 5.8 mg | | | |
| 0020 | 1:4 | 68% | 13% | 10% | 8.6% |
| | | 0.8 mg | | | |

| | | harvest | | | | | |
|---|---|---|---|---|---|---|---|
| | | 3-liter | | | | | |
| | | after | | | | | |
| | | preparative | | | | | |
| | | protein A | | | | | |
| | | purification | by-products | | end | by-products | |
| | vector | monomer | SEC [%] | | product | SEC [%] | |
| antibody | ratio | SEC | HMW | LMW | SEC | HMW | LMW |
| 0012 | 1:1:3 | 78% | 0 | 22 | 97.5% | 0 | 2.5 |
| 0024 | 1:1:3 | 80% | 0 | 20 | 96% | 0 | 4 |
| 0015 | 1:1:3 | 87% | 0 | 13 | 97% | 0 | 3 |
| 0020 | 1:4 | 53% | 7 | 40 | 97% | 0 | 3 |

With this approach a production cell line can be obtained that produces the heterodimeric antibody with an improved product profile, i.e. with increased product and reduced product-related impurities.

The co-transfection with an expression plasmid comprising a sole antibody chain expression cassette for the light chain was used for the generation of stable production cell lines.

CHO-K1 cells were transfected at a plasmid ratio of 1(LC):1(LC+HChole):3(LCcross+HCknob). Cells that had stably integrated the foreign DNA into their genome were selected with methotrexate. Stable cell lines were isolated and evaluated in a four-day batch culture with regard product quality. Product was isolated using protein A affinity chromatography and analyzed with CE-SDS.

relative peak area [%]
(non-reduced analysis)

| | | | hole- | |
|---|---|---|---|---|
| | ½ mAb | ½ mAb | hole | antibody |
| cell line | hole | knob | chain dimer | monomer |
| 0130 | 3 | 0 | 3 | 94 |
| 0117 | 2 | 2 | 4 | 92 |
| 0147 | 0 | 13 | 0 | 88 |
| 0185 | 3 | 1 | 7 | 88 |
| 0097 | 2 | 6 | 9 | 84 |

### Bispecific, Trivalent Anti-Human CD20/Human Transferrin Receptor Antibody

This antibody is a bispecific antibody consisting of a full-length core antibody and a fused Fab fragment in which certain domains are crosswise exchanged. Thus, the resulting bispecific antibody is asymmetric. Therefore, the bispecific antibody is produced using the heterodimerization technology called knobs-into-holes using a first heavy chain with the so-called knob mutations (HCknob) and a second heavy chain with the so-called hole mutations (HChole).

In this example a co-transfection has been used.

Antibody 0039, antibody 0041, antibody 0040 and antibody 0042 are reported in WO 2017/055542 A1 (SEQ ID NO: 06 to 09, SEQ ID NO: 01 to 03 and SEQ ID NO: 10, SEQ ID NO: 11 to 13 and SEQ ID NO: 22 and SEQ ID NO: 14 to 17 of WO 2017/055542 A1, respectively).

Antibody 0038 is a full length antibody comprising one heavy chain with the hole mutations and one heavy chain with the knob mutations, wherein to the C-terminus of the heavy chain with the knob mutations the VL of a scFab is fused via a linker. Both normal Fab arms have been modified to comprise charges to assist correct assembly.

Antibody 0039 is a full length antibody comprising one heavy chain with the hole mutations and one heavy chain with the knob mutations, wherein to the C-terminus of the heavy chain with the knob mutations the VL of a Fab is fused via a linker, wherein in the Fab the VH and VL domains are exchanged (VH-VL domain crossover). Both Fabs with unchanged domains have been modified to comprise charges to assist correct assembly.

Antibody 0041 is a full length antibody comprising one heavy chain with the hole mutations and one heavy chain with the knob mutations, wherein to the C-terminus of the heavy chain with the knob mutations the VH of a Fab is fused via a linker, wherein in the Fab the CH1 and CL domains are exchanged (CH-CL domain crossover). Both pairs of heavy and light chains of the full length antibody have been modified to comprise charges to assist correct assembly as well as the Fab. Both Fabs with unchanged domains have been modified to comprise charges to assist correct assembly.

Antibody 0040 is a full length antibody comprising one heavy chain with the hole mutations and one heavy chain with the knob mutations, wherein to the C-terminus of the heavy chain with the knob mutations the VH of a Fab is fused via a linker, wherein in the Fab the CH1 and CL domains are exchanged (CH-CL domain crossover).

Antibody 0042 is a full length antibody comprising one heavy chain with the hole mutations and one heavy chain with the knob mutations, wherein to the heavy chain with the knob mutations the CH1 of a Fab is fused via a linker to the N-terminus, wherein in the Fab of the fused heavy chain the VH and VL domains are exchanged (VH-VL domain crossover). Both Fabs with unchanged domains have been modified to comprise charges to assist correct assembly.

Different allocation/combination of the respective polypeptides on different expression vectors, different ratios of the resulting vectors and different transfection sequences have been used for the recombinant production of the bi specific antibodies.

LC+HChole: expression vector comprising one expression cassette for the heavy chain with the hole mutation and the light chain.

LCcross+HCknob: expression vector comprising one expression cassette for the heavy chain with the knob mutation and the light chain with domain crossover.

LC: expression vector comprising one expression cassette for the light chain.

LCcross: expression vector comprising one expression cassette for the light chain with domain crossover.

HCknob: expression vector comprising one expression cassette for the heavy chain with knob mutation and a fused scFab.

Different allocation/combination of the respective polypeptides on different expression vectors and different ratios of the resulting vectors have been used for the recombinant production of the bispecific antibodies in HEK cells. The results are presented in the following Table.

| | | | relative peak area | | |
|---|---|---|---|---|---|
| | | | (non-reduced) [%] | | |
| | | | | hole- | |
| | | | | hole | |
| | | | ½ mAb | chain | antibody |
| antibody | vector ratio for transfection | | hole | dimer | monomer |
| 0039 | 1:3 | LC + | 9 | 10 | 80 |
| 0039 | 1:4 | HChole:LCcross + | 6 | 6 | 88 |
| | | HCknob | | | |
| 0039 | 1:3:2 | LC + | 17 | 9 | 74 |
| 0039 | 1:4:2 | HChole:LCcross + | 10 | 5 | 85 |
| | | HCknob:LCcross | | | |
| 0039 | 1:1:3 | LC:LC + | 6 | 6 | 88 |
| 0039 | 1:1:4 | HChole:LCcross + | 3 | 4 | 93 |
| | | HCknob | | | |
| 0040 | 1:3 | LC + | 10 | 18 | 72 |
| 0040 | 1:4 | HChole:LCcross + | 6 | 7 | 87 |
| | | HCknob | | | |
| 0040 | 1:3:2 | LC + HChole:LCcross + | 5 | 7 | 89 |
| 0040 | 1:4:2 | HCknob:LCcross | 3 | 5 | 92 |
| 0040 | 1:1:3 | LC:LC + | 16 | 48 | 35 |
| 0040 | 1:1:4 | HChole:LCcross + | 6 | 23 | 69 |
| | | HCknob | | | |
| 0041 | 1:3 | LC + | 3 | 5 | 92 |
| 0041 | 1:4 | HChole:LCcross + | 1 | 2 | 97 |
| | | HCknob | | | |
| 0041 | 1:3:2 | LC + | — | 2 | 98 |
| 0041 | 1:4:2 | HChole:LCcross + | — | 1 | 99 |
| | | HCknob:LCcross | | | |
| 0041 | 1:1:3 | LC:LC + | — | 3 | 97 |
| 0041 | 1:1:4 | HChole:LCcross + | — | 2 | 97 |
| | | HCknob | | | |
| 0042 | 1:2 | LC + | — | 1 | 99 |
| | | HC-hole:HCknob | | | |

Different allocation/combination of the respective polypeptides on different expression vectors and different ratios of the resulting vectors have been used for the recombinant production of the bispecific antibodies in CHO-K1 cells. The results are presented in the following Table.

| | | | relative peak area | | | | |
|---|---|---|---|---|---|---|---|
| | | | (non-reduced, CE-SDS) [%] | | | | |
| | vector ratio for | | ½ mAb | ½ mAb | hole-hole | | antibody |
| antibody | transfection | | hole | knob | chain dimer | LC | monomer |
| 0038 | 1:4 | LC + | 5 | 4 | 13 | 1 | 54 |
| | | HChole:HCknob | | | | | |
| 0039 | 1:4:2 | LC + | 0 | 10 | 5 | 2 | 79 |
| 0039 | 1:3:2 | HChole:LCcross + | 0 | 9 | 5 | 2 | 80 |
| | | HCknob:LCcross | | | | | |
| 0040 | 1:2:1 | LC + | 10 | 1 | 15 | 3 | 68 |
| 0040 | 1:3:2 | HChole:LCcross + | 2 | 4 | 7 | 3 | 79 |
| | | HCknob:CrossLC | | | | | |
| 0041 | 1:4:2 | LC + | 1 | 3 | 6 | 1 | 86 |
| 0041 | 1:3:2 | HC-hole:LCcross + | 3 | 1 | 7 | 1 | 85 |
| | | HCknob:CrossLC | | | | | |
| 0042 | 1:2 | LC + | 4 | 1 | 4 | 3 | 86 |
| | | HC-hole:CrossLC + | | | | | |
| | | HCknob | | | | | |

The bispecific antibodies have been produced in small scale in CHO-S cells and the by-product distribution has been analyzed after a first purification step using a protein A affinity chromatography and after the second purification step using a preparative size-exclusion chromatography. The results are presented in the following Table.

| | | | by-product distribution | | | |
|---|---|---|---|---|---|---|
| | | harvest 3-liter | (CE-SDS not red.) | | | |
| | | fermentation after | | | | hole-hole |
| | | protein A | | | | dimer + |
| | vector | product monomer | | | ½ mAb | ½ mAb |
| antibody | ratio | (CE-SDS not red./yield) | LC | HC hole | hole | knob |
| 0039 | 1:3:2 | 55% | 16 | 6 | 11 | 12 |
| | | 26 mg | | | | |
| 0040 | 1:3:2 | 44% | 23 | 8 | 7 | 17 |
| | | 74.5 mg | | | | |
| 0041 | 1:4:2 | 82% | 11 | 0 | 0 | 7 |
| | | >80 mg | | | | |
| 0042 | 1:2 | 83% | 9 | 0 | 0 | 8 |
| | | 68.4 mg | | | | |

| | | harvest 3-liter | by-product distribution | | | |
|---|---|---|---|---|---|---|
| | | fermentation after | (CE-SDS not red.) | | | |
| | | protein A and | | | | hole-hole |
| | | preparative SEC | | | | dimer + |
| | vector | product monomer | | | ½ mAb | ½ mAb |
| antibody | ratio | (CE-SDS not red./yield) | LC | HC hole | hole | knob |
| 0039 | 1:4 | 8.2 mg | 10 | 0 | 2 | 15 |
| | | 73% | | | | |
| 0040 | | 29.7 mg | 10 | 0 | 0 | 14 |
| | | 77% | | | | |
| 0041 | 1:4:2 | >44 mg | 8 | 0 | 0 | 13 |
| | | 79% | | | | |
| 0042 | 1:2 | 43.6 mg | 3 | 0 | 0 | 3 |
| | | >90% | | | | |

| | | harvest 3-liter | | | | | |
|---|---|---|---|---|---|---|---|
| | | after protein A | by-products | | end | by-products | |
| | vector | purification | SEC [%] | | product | SEC [%] | |
| antibody | ratio | monomer SEC | HMW | LMW | SEC | HMW | LMW |
| 0039 | | 90% | 2 | 7 | 95% | 0 | 5 |
| 0040 | | 89% | 5 | 6 | 96.5% | 1 | 2.5 |
| 0041 | 1:4:2 | 94% | 6 | 0 | 97.5% | 0.5 | 2 |
| 0042 | 1:2 | 95% | 2 | 3 | 97% | 1 | 2 |

The bispecific antibodies have been produced in different cell lines. The results are shown in the following Table.

| | | | harvest 3- | end product |
|---|---|---|---|---|
| | | | liter after | (preparative |
| | | | protein A | protein A |
| | micro purification | | purification | and preparative |
| | with protein A | | monomer | SEC purification) |
| | product monomer | | (yield/CE- | (yield/CE- |
| | (CE-SDS) | | SDS not red.) | SDS not red.) |
| antibody | CHO-K1 | HEK293 | CHO-S | CHO-S |
| 0039 | 80% | 93% | 7.4 mg/l | 8.2 mg |
| | | | 55% | 73% |
| 0040 | 83% | 92% | 21.3 mg/l | 29.7 mg |
| | | | 44% | 77% |
| 0041 | 85% | 99% | >21 mg/l | >44 mg |
| | | | 82% | 79% |
| 0042 | 91% | 99% | 19 mg/l | 43.8 mg |
| | | | 83% | 94% |

With this approach a production cell line can be obtained that produces the heterodimeric antibody with an improved product profile, i.e. with increased product and reduced product-related impurities.

### Bispecific, Bivalent Anti-Human PD1/Human Tim3 Antibody

This antibody is a bispecific antibody consisting of a full-length antibody with knob-into-hole mutations in the Fc-region and an artificial disulfide bridge between the CH3 domains, in which in the heavy and light chain pair forming the binding site for PD1 the VH and VL domains are replaced by each other. Thus, the resulting bispecific antibody is asymmetric. Therefore, the bispecific antibodies are produced using the heterodimerization technology called knobs-into-holes using a first heavy chain with the so-called knob mutations (HCknob) and a second heavy chain with the so-called hole mutations (HChole). For sequences see WO 2017/055404 A1.

In this example a co-transfection has been used.

Here several different versions of expression plasmids were combined to generate a cell line expressing the above antibody. These approaches differ in the combination of plasmids, but not in the antibody.

For the 0516 transfection vector 1 comprising expression cassettes for the first light chain (LC-1) and the first heavy chain with the hole mutations (HC-1-hole) of the IgG 1 subclass and vector 2 comprising expression cassettes for the domain exchanged second light chain (CrossLC-2) and the domain exchanged second heavy chain with the knob mutations (CrossHC-2-knob) of the IgG1 subclass were co-transfected at a 1:1 ratio.

For the 0517 transfection vector 1 comprising expression cassettes for the first light chain (LC-1) and the first heavy chain with the hole mutations (HC-1-hole) of the IgG 1 subclass, vector 2 comprising expression cassettes for the domain exchanged second light chain (CrossLC-2) and the domain exchanged second heavy chain with the knob mutations (CrossHC-2-knob) of the IgG1 subclass, and vector 3 comprising an expression cassette for the domain exchanged second light chain (CrossLC-2) were co-transfected at a 1:1:1 ratio.

For the 0518 transfection vector 1 comprising expression cassettes for the domain exchanged second light chain (CrossLC-2) and the first heavy chain with the hole mutations (HC-1-hole) of the IgG 1 subclass and vector 2 comprising expression cassettes for the first light chain (LC-1) and the domain exchanged second heavy chain with the knob mutations (CrossHC-2-knob) of the IgG1 subclass were co-transfected at a 1:1 ratio.

For the 0519 transfection vector 1 comprising expression cassettes for the domain exchanged second light chain (CrossLC-2) and the first heavy chain with the hole mutations (HC-1-hole) of the IgG 1 subclass, vector 2 comprising expression cassettes for the first light chain (LC-1) and the domain exchanged second heavy chain with the knob mutations (CrossHC-2-knob) of the IgG1 subclass, and vector 3 comprising an expression cassette for the domain exchanged second light chain (CrossLC-2) were co-transfected at a 1:1:1 ratio. The results are presented in the following Table.

The correctly assemble antibody has a stoichiometry of ABCD with A=second heavy chain with the knob mutations (CrossHC-2-knob) of the IgG1 subclass, B=the first heavy chain with the hole mutations (HC-1-hole) of the IgG 1 subclass, C=the domain exchanged second light chain (CrossLC-2), and D=the first light chain (LC-1).

The main compound-related side products formed were wrongly assembled antibodies. The two main by-products were both four chain antibodies. The first one was a hetero-hole-knob-HC dimer in which the crossed light chain was replaced by the non-crossed light chain (ABD2). The second one was a homo-hole-hole half antibody dimer (B2D2).

It can be seen that for transfections employing an additional plasmid comprising as only expression cassette that for the domain exchanged light chain improved results, i.e. less product-related side products are present, can be obtained (see FIGS. 1A to 1D).

| | | | relative peak area | |
|---|---|---|---|---|
| | | | (non-reduced) | |
| | | | by- | by- |
| | | | product 1 | product 2 |
| transfection | vector ratio for transfection | | (ABD2) | (B2D2) |
| 0516 | 1:1 | LC-1 + | ++ | +++ |
| | | HC-1-hole:CrossLC-2 + | | |
| | | CrossHC-2-knob | | |
| 0517 | 1:1:1 | LC-1 + | no | ++ |
| | | HC-1-hole:CrossLC-2 + | | |
| | | HC-2-knob:CrossLC-2 | | |
| 0518 | 1:1 | CrossLC-2 + | +++++ | + |
| | | HC-1-hole:LC-1 + | | |
| | | CrossHC-2-knob | | |
| 0519 | 1:1:1 | CrossLC-2 + | + | no |
| | | HC-1-hole:LC-1 + | | |
| | | HC-2-knob:CrossLC-2 | | |

As can be seen from FIG. 1 the product-related side-products, especially the ABD2 side product, can be reduced. This concomitantly reduces the product loss during the subsequent purification steps. For example, either the number of needed purification steps can be reduced or the loss of product due to overlapping peaks and fractionation can be reduced (the peaks are more separated and thereby can be cut with reduced loss of product) or both. Thereby the obtainable yield can be increased.

With this approach a production cell line can be obtained that produces the heterodimeric antibody with an improved product profile, i.e. with increased product and reduced product-related impurities.

### Bispecific, Tetravalent Anti-Human FAP/DR5 Antibody

Bispecific FAP-DR5 antibodies were generated by fusion of a FAP binding domain to the DR5 IgG heavy chain at the C-terminus via a (G4S)4 linker (SEQ ID NO: 1). The DR5 portion consisted of the variable-light chain (VL) and the variable-heavy chain (VH) of drozitumab (see US 2007/003141401) or novel DR5 antibodies generated by phage display. To minimize light-chain mispairing side-products, the CrossMab technology with domain crossover was used. The FAP-binding unit was engineered as a crossed Fab in which the VH was fused to the constant light chain (CL) and the VL to a CH1 (constant-heavy 1) domain. For sequences see WO 2016/055432.

In this example a sequential transfection has been used.

The respective polypeptide expression cassettes were distributed on different expression vectors.

LC+HChole: expression vector comprising one expression cassette for the heavy chain with the hole mutation and the light chain.

LCcross+HCknob: expression vector comprising one expression cassette for the heavy chain with the knob mutation and the light chain with domain crossover.

LC: expression vector comprising one expression cassette for the light chain.

LCcross: expression vector comprising one expression cassette for the light chain with domain crossover.

Clone 131 was obtained by standard two-plasmid transfection each comprising two expression cassettes for the expression of a bispecific antibody (full length antibody with one CH1/CL cross-Fabs attached to each C-terminus of the heavy chains).

This clone produced the following composition.

| | | | relative peak area (SEC) [%] | | | |
|---|---|---|---|---|---|---|
| | | vector ratio original | mispaired | | mispaired | |
| | product | transfection LC + | product | | product | |
| | concentration | HChole:LCcross + | with 3xLC | | with 3xLC | 5/6 |
| clone | [μg/ml] | HCknob | FAP | monomer | DR5 | antibody |
| 131 | 1750 | 1:3 | 5.4 | 75.6 | 8.5 | 10.6 |

This clone has been used as the basic clone for a second transfection with a plasmid comprising only the cross light chain of the FAP binding site.

The characteristics of some Exemplary resulting clones are shown in the following Table.

| | | vector ratio (original | relative peak area [%] | | | |
|---|---|---|---|---|---|---|
| | | transfection):additional | mispaired | | mispaired | |
| | product | transfection (LC + | product | | product | |
| | concentration | HChole:LCcross + | with 3xLC | | with 3xLC | 5/6 |
| clone | [μg/ml] | HCknob):LCcross | FAP | monomer | DR5 | antibody |
| 131 | 1750 | (1:3):0 - | 5.4 | 75.6 | 8.5 | 10.6 |
| | | reference | | | | |
| 368 | 1429 | (1:3):1 | 0.35 | 91.45 | 6.21 | — |
| 449 | 1571 | (1:3):1 | 0.41 | 90.99 | 7.11 | — |
| 485 | 1999 | (1:3):1 | 0.35 | 90.45 | 7.5 | — |
| 499 | 1689 | (1:3):1 | 0.28 | 91.23 | 6.86 | — |
| av. | | | 0.96 +/− | 87.01 +/− | 9.45 +/− | 2.81 +/− |
| | | | 1.35 | 3.67 | 2.16 | 4.02 |

The CE-SDS results are presented in the following Table (231=5/6 antibody; 242=monomer) and FIG. 2.

| | Rel Peak Area | | | | | | |
|---|---|---|---|---|---|---|---|
| | No | | | | | | |
| clone | 140 | 193 | 207 | 210 | 231 | 252 | 280 |
| 0499 | 0 | | 3 | 1 | 3 | 92 | |
| 0496 | 1 | | 3 | 2 | 4 | 91 | 0 |
| 0492 | 0 | | 4 | 1 | 3 | 89 | 2 |
| 0485 | 0 | 0 | 3 | 2 | 2 | 92 | 0 |
| 0474 | 0 | 0 | 3 | 2 | 4 | 90 | 1 |
| 0473 | 1 | | 3 | 2 | 4 | 91 | 0 |
| 0449 | 1 | 0 | 3 | 2 | 3 | 91 | |
| 0448 | 1 | 0 | 4 | 2 | 6 | 87 | 1 |
| 0370 | 0 | 0 | 4 | 3 | 14 | 79 | 0 |
| 0368 | 0 | | 2 | 1 | 4 | 92 | 0 |
| 0362 | 0 | | 4 | 1 | 3 | 90 | 1 |
| 0361 | 0 | | 3 | 1 | 3 | 91 | 1 |
| 0358 | 1 | 0 | 3 | 2 | 12 | 81 | 0 |
| 0354 | 1 | 0 | 4 | 2 | 4 | 90 | 0 |
| 0352 | 1 | | 3 | 2 | 4 | 91 | 0 |
| 0350 | 1 | | 3 | 1 | 4 | 88 | 2 |
| 0348 | 0 | | 3 | 1 | 3 | 92 | 0 |
| 0345 | 0 | 0 | 4 | 1 | 3 | 90 | 1 |
| 0342 | 0 | 0 | 4 | 2 | 3 | 89 | 1 |
| 0341 | 0 | 0 | 4 | 1 | 4 | 89 | 1 |
| 0339 | 1 | 0 | 4 | 1 | 4 | 89 | 1 |
| 0336 | 1 | 0 | 4 | 1 | 5 | 88 | 1 |
| 0334 | 1 | | 3 | 2 | 4 | 91 | 0 |
| 0131 | 1 | 1 | 4 | 4 | 21 | 69 | 0 |

With this approach a production cell line can be obtained that produces the heterodimeric antibody with an improved product profile, i.e. with increased product and reduced product-related impurities.

### General Recombinant Methods and Compositions for Producing Antibodies

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the second light chain comprising the first heteromonomeric Fc-region polypeptide, one for the second light chain, and one for the second heavy chain comprising the second heteromonomeric Fc-region polypeptide. For example, one of the heterodimeric heavy chain comprises to so-called “knobs mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73). Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs and T-cell bispecific antibodies.

In one embodiment isolated nucleic acids encoding an antibody as used in the methods as reported herein are provided.

In a further embodiment, one or more vectors (e.g., expression vectors) comprising such nucleic acid(s) are provided.

In a further embodiment, a host cell comprising such nucleic acid(s) is provided.

In one such embodiment, a host cell comprises (e.g., has been transformed with):
in case of a native antibody or native antibody fragment:
   (1) a vector comprising a nucleic acid that encodes an amino acid sequence comprising the VL of the antibody and an amino acid sequence comprising the VH of the antibody, or
   (2) a first vector comprising a nucleic acid that encodes an amino acid sequence comprising the VL of the antibody and a second vector comprising a nucleic acid that encodes an amino acid sequence comprising the VH of the antibody.
in case of a bispecific antibody with heterodimeric heavy chains:
   (1) a first vector comprising a first pair of nucleic acids that encode amino acid sequences one of them comprising the first VL and the other comprising the first VH of the antibody and a second vector comprising a second pair of nucleic acids that encode amino acid sequences one of them comprising the second VL and the other comprising the second VH of the antibody, or
   (2) a first vector comprising a first nucleic acid that encode an amino acid sequence comprising one of the variable domains (preferably a light chain variable domain), a second vector comprising a pair of nucleic acids that encode amino acid sequences one of them comprising a light chain variable domain and the other comprising the first heavy chain variable domain, and a third vector comprising a pair of nucleic acids that encode amino acid sequences one of them comprising the respective other light chain variable domain as in the second vector and the other comprising the second heavy chain variable domain, or
   (3) a first vector comprising a nucleic acid that encodes an amino acid sequence comprising the first VL of the antibody, a second vector comprising a nucleic acid that encodes an amino acid sequence comprising the first VH of the antibody, a third vector comprising a nucleic acid that encodes an amino acid sequence comprising the second VL of the antibody, and a fourth vector comprising a nucleic acid that encodes an amino acid sequence comprising the second VH of the antibody.

In one embodiment, the host cell is eukaryotic, e.g. a Chinese Hamster Ovary (CHO) cell or lymphoid cell (e.g., Y0, NS0, Sp20 cell). In one embodiment, a method of making an anti-[[PRO]] antibody is provided, wherein the method comprises culturing a host cell comprising nucleic acids encoding the antibody, as provided above, under conditions suitable for expression of the antibody, and optionally recovering the antibody from the host cell (or host cell culture medium).

For recombinant production of an anti-[[PRO]] antibody, nucleic acids encoding an antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, N.J. (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp.

### DESCRIPTION OF THE FIGURES

FIGS. 1A-D: Deglycosylated ESI-MS total ion chromatogram for
1A transfection 0516;
1B transfection 0517;
1C transfection 0518;
1D transfection 0519;
1A: second heavy chain with the knob mutations (CrossHC-2-knob) of the IgG1 subclass; 1B: the first heavy chain with the hole mutations (HC-1-hole) of the IgG 1 subclass; 1C: the domain exchanged second light chain (CrossLC-2), 1D: the first light chain (LC-1).

FIGS. 2A-B: Relative monomer content 2A and 5/6 antibody side product 2B of the reference clone 0131 and the clones obtained with the method as described herein determined by CE-SDS.

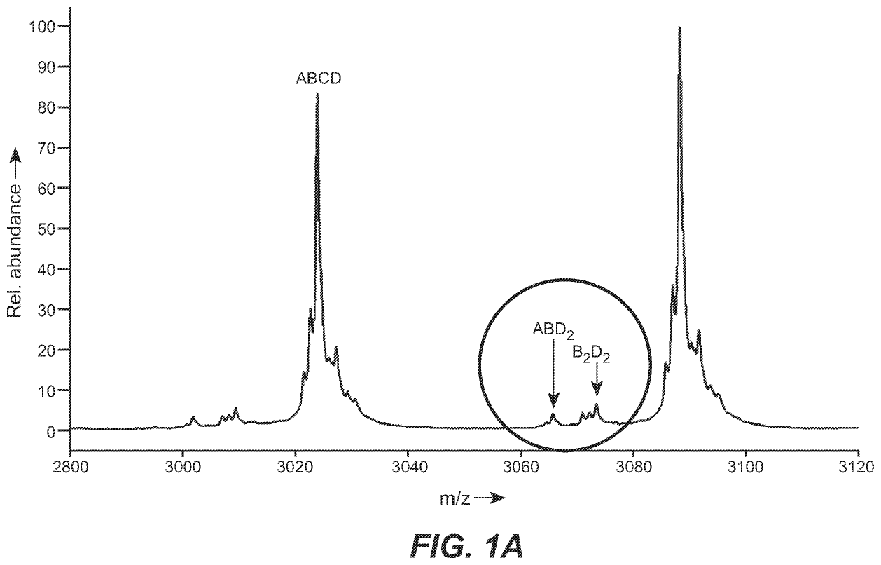

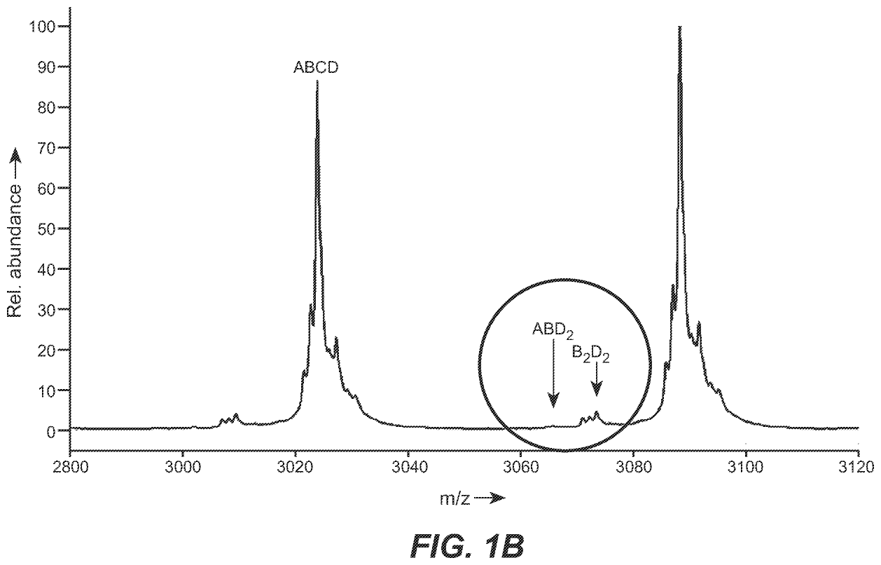

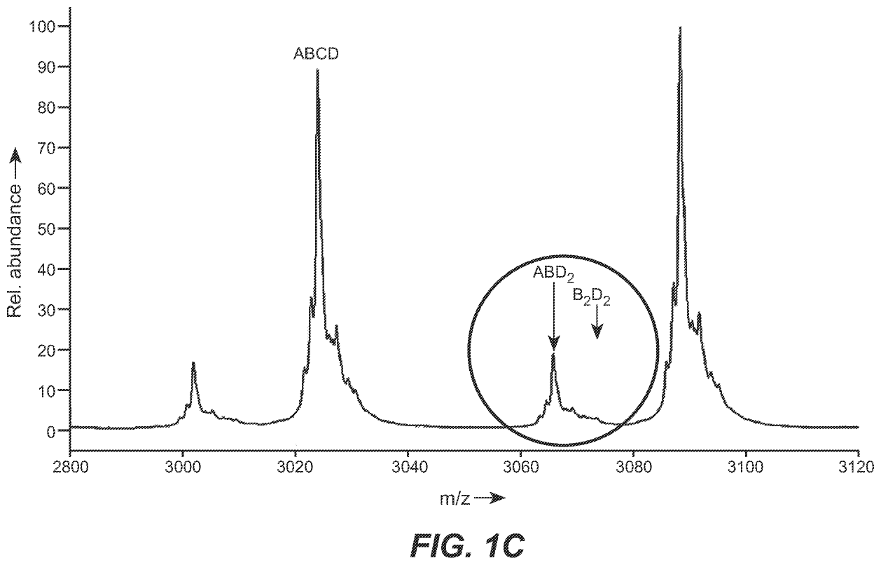

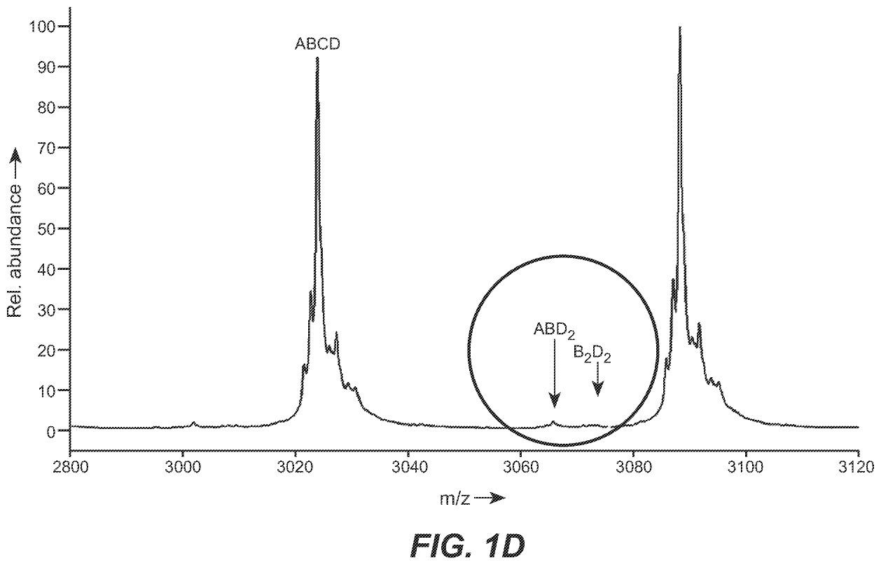

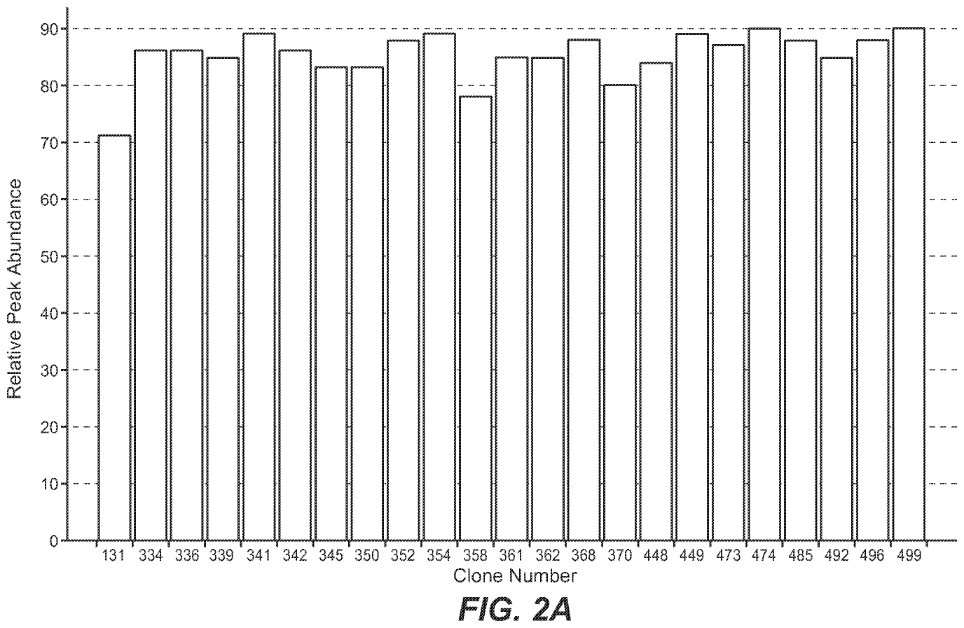

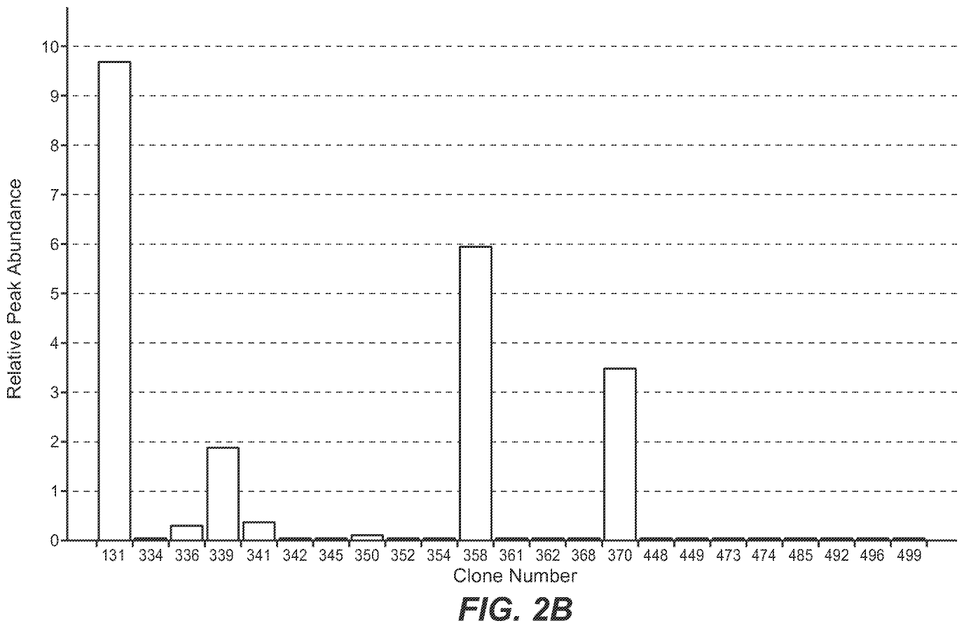

### EXAMPLES

The following are examples of methods and compositions of the invention. It is understood that various other embodiments may be practiced, given the general description provided above.

### Materials & General Methods

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to numbering according to Kabat (Kabat, E. A., et al., Sequences of Proteins of Immunological Interest, 5th ed., Public Health Service, National Institutes of Health, Bethesda, Md. (1991)).

### Recombinant DNA Techniques

Standard methods were used to manipulate DNA as described in Sambrook, J. et al., Molecular Cloning: A laboratory manual; Cold Spring Harbor Laboratory Press, Cold Spring Harbor, N.Y., 1989. The molecular biological reagents were used according to the manufacturer's instructions.

### Gene Synthesis

Desired gene segments were prepared from oligonucleotides made by chemical synthesis. The long gene segments, which were flanked by singular restriction endonuclease cleavage sites, were assembled by annealing and ligating oligonucleotides including PCR amplification and subsequently cloned via the indicated restriction sites. The DNA sequences of the subcloned gene fragments were confirmed by DNA sequencing. Gene synthesis fragments were ordered according to given specifications at Geneart (Regensburg, Germany).

### DNA Sequence Determination

DNA sequences were determined by double strand sequencing performed at MediGenomix GmbH (Martinsried, Germany) or SequiServe GmbH (Vaterstetten, Germany).

### DNA and Protein Sequence Analysis and Sequence Data Management

The GCG's (Genetics Computer Group, Madison, Wis.) software package version 10.2 and Infomax's Vector NT1 Advance suite version 8.0 was used for sequence creation, mapping, analysis, annotation and illustration.

### Expression Vectors

For the expression of the described bispecific antibodies, expression vectors for transient expression (e.g. in HEK293 cells) based either on a cDNA organization with or without a CMV-intron A promoter or on a genomic organization with a CMV promoter can be applied.

Beside the antibody expression cassette, the vectors contain:
an origin of replication which allows replication of this vector in E. coli, and
a ß-lactamase gene which confers ampicillin resistance in E. coli.

The transcription unit of the antibody gene is composed of the following elements:
unique restriction site(s) at the 5′ end
the immediate early enhancer and promoter from the human cytomegalovirus,
the intron A sequence in the case of cDNA organization,
a 5′-untranslated region derived from a human antibody gene,
an immunoglobulin heavy chain signal sequence,
the respective antibody chain encoding nucleic acid either as cDNA or with genomic exon-intron organization,
a 3′ untranslated region with a polyadenylation signal sequence, and
unique restriction site(s) at the 3′ end.

The fusion genes encoding the antibody chains are generated by PCR and/or gene synthesis and assembled by known recombinant methods and techniques by connection of the according nucleic acid segments e.g. using unique restriction sites in the respective vectors. The subcloned nucleic acid sequences are verified by DNA sequencing. For transient transfections larger quantities of the vectors are prepared by vector preparation from transformed E. coli cultures (Nucleobond AX, Macherey-Nagel).

For all constructs knob-into-hole heterodimerization technology was used with a typical knob (T366W) substitution in the first CH3 domain and the corresponding hole substitutions (T366S, L368A and Y407V) in the second CH3 domain (as well as two additional introduced cysteine residues S354C/Y349′C) (contained in the respective corresponding heavy chain (HC) sequences depicted above).

### Cell Culture Techniques

Standard cell culture techniques as described in Current Protocols in Cell Biology (2000), Bonifacino, J. S., Dasso, M., Harford, J. B., Lippincott-Schwartz, J. and Yamada, K. M. (eds.), John Wiley & Sons, Inc., are used.

### Transient Transfections in HEK293-F System

The bispecific antibodies are produced by transient expression. Therefore, a transfection with the respective vectors using the HEK293-F system (Invitrogen) according to the manufacturer's instruction is done. Briefly, HEK293-F cells (Invitrogen) growing in suspension either in a shake flask or in a stirred fermenter in serum-free FreeStyle™ 293 expression medium (Invitrogen) are transfected with a mix of the respective expression vectors and 293fectin™ or fectin (Invitrogen). For 2 L shake flask (Corning) HEK293-F cells are seeded at a density of 1.0*10⁶ cells/mL in 600 mL and incubated at 120 rpm, 8% CO₂. On the next day the cells are transfected at a cell density of approx. 1.5*10⁶ cells/mL with approx. 42 mL of a mixture of A) 20 mL Opti-MEM medium (Invitrogen) comprising 600 μg total vector DNA (1 μg/mL) and B) 20 ml Opti-MEM medium supplemented with 1.2 mL 293 fectin or fectin (2 μl/mL). According to the glucose consumption glucose solution is added during the course of the fermentation. The supernatant containing the secreted antibody is harvested after 5-10 days and antibodies are either directly purified from the supernatant or the supernatant is frozen and stored.

### Protein Determination

The protein concentration of purified antibodies and derivatives was determined by determining the optical density (OD) at 280 nm, using the molar extinction coefficient calculated on the basis of the amino acid sequence according to Pace, et al., Protein Science 4 (1995) 2411-1423.

### Antibody Concentration Determination in Supernatants

The concentration of antibodies and derivatives in cell culture supernatants was estimated by immunoprecipitation with protein A agarose-beads (Roche Diagnostics GmbH, Mannheim, Germany). Therefore, 60 μL protein A Agarose beads were washed three times in TBS-NP40 (50 mM Tris buffer, pH 7.5, supplemented with 150 mM NaCl and 1% Nonidet-P40). Subsequently, 1-15 mL cell culture supernatant was applied to the protein A Agarose beads pre-equilibrated in TBS-NP40. After incubation for at 1 hour at room temperature the beads were washed on an Ultrafree-MC-filter column (Amicon) once with 0.5 mL TBS-NP40, twice with 0.5 mL 2× phosphate buffered saline (2×PBS, Roche Diagnostics GmbH, Mannheim, Germany) and briefly four times with 0.5 mL 100 mM Na-citrate buffer (pH 5.0). Bound antibody was eluted by addition of 35 μl NuPAGE® LDS sample buffer (Invitrogen). Half of the sample was combined with NuPAGE® sample reducing agent or left unreduced, respectively, and heated for 10 min at 70° C. Consequently, 5-30 μl were applied to a 4-12% NuPAGE® Bis-Tris SDS-PAGE gel (Invitrogen) (with MOPS buffer for non-reduced SDS-PAGE and MES buffer with NuPAGE® antioxidant running buffer additive (Invitrogen) for reduced SDS-PAGE) and stained with Coomassie Blue.

The concentration of the antibodies in cell culture supernatants was quantitatively measured by affinity HPLC chromatography. Briefly, cell culture supernatants containing antibodies that bind to protein A were applied to an Applied Biosystems Poros A/20 column in 200 mM KH₂PO₄, 100 mM sodium citrate, pH 7.4 and eluted with 200 mM NaCl, 100 mM citric acid, pH 2.5 on an Agilent HPLC 1100 system. The eluted antibody was quantified by UV absorbance and integration of peak areas. A purified standard IgG1 antibody served as a standard.

Alternatively, the concentration of antibodies and derivatives in cell culture supernatants was measured by Sandwich-IgG-ELISA. Briefly, StreptaWell High Bind Streptavidin A-96 well microtiter plates (Roche Diagnostics GmbH, Mannheim, Germany) were coated with 100 μL/well biotinylated anti-human IgG capture molecule F(ab′)2<h-Fcγ>BI (Dianova) at 0.1 μg/mL for 1 hour at room temperature or alternatively overnight at 4° C. and subsequently washed three times with 200 μL/well PBS, 0.05% Tween (PBST, Sigma). Thereafter, 100 μL/well of a dilution series in PBS (Sigma) of the respective antibody containing cell culture supernatants was added to the wells and incubated for 1-2 hour on a shaker at room temperature. The wells were washed three times with 200 μL/well PBST and bound antibody was detected with 100 μl F(ab′)2<hFcγ>POD (Dianova) at 0.1 μg/mL as the detection antibody by incubation for 1-2 hours on a shaker at room temperature. Unbound detection antibody was removed by washing three times with 200 μL/well PBST. The bound detection antibody was detected by addition of 100 μL ABTS/well followed by incubation. Determination of absorbance was performed on a Tecan Fluor Spectrometer at a measurement wavelength of 405 nm (reference wavelength 492 nm).

### Preparative Antibody Purification

Antibodies were purified from filtered cell culture supernatants referring to standard protocols. In brief, antibodies were applied to a protein A Sepharose column (GE healthcare) and washed with PBS. Elution of antibodies was achieved at pH 2.8 followed by immediate neutralization. Aggregated protein was separated from monomeric antibodies by size exclusion chromatography (Superdex 200, GE Healthcare) in PBS or in 20 mM Histidine buffer comprising 150 mM NaCl (pH 6.0). Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® antioxidant running buffer additive) or MOPS (non-reduced gels) running buffer was used.

### CE-SDS

Purity and antibody integrity were analyzed by CE-SDS using microfluidic Labchip technology (PerkinElmer, USA). Therefore, 5 μl of antibody solution was prepared for CE-SDS analysis using the HT Protein Express Reagent Kit according manufacturer's instructions and analyzed on LabChip GXII system using a HT Protein Express Chip. Data were analyzed using LabChip GX Software.

### Analytical Size Exclusion Chromatography

Size exclusion chromatography (SEC) for the determination of the aggregation and oligomeric state of antibodies was performed by HPLC chromatography. Briefly, protein A purified antibodies were applied to a Tosoh TSKgel G3000SW column in 300 mM NaCl, 50 mM KH₂PO₄/K₂HPO₄ buffer (pH 7.5) on an Dionex Ultimate® system (Thermo Fischer Scientific), or to a Superdex 200 column (GE Healthcare) in 2×PBS on a Dionex HPLC-System. The eluted antibody was quantified by UV absorbance and integration of peak areas. BioRad Gel Filtration Standard 151-1901 served as a standard.

### Mass Spectrometry

This section describes the characterization of the bispecific antibodies with emphasis on their correct assembly. The expected primary structures were analyzed by electrospray ionization mass spectrometry (ESI-MS) of the deglycosylated intact antibody and in special cases of the deglycosylated/limited LysC digested antibody.

The antibodies were deglycosylated with N-Glycosidase F in a phosphate or Tris buffer at 37° C. for up to 17 h at a protein concentration of 1 mg/ml. The limited LysC (Roche Diagnostics GmbH, Mannheim, Germany) digestions were performed with 100 μg deglycosylated antibody in a Tris buffer (pH 8) at room temperature for 120 hours, or at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Example 1

### Expression and Purification

The bispecific antibodies were produced as described above in the general materials and methods section.

The bispecific antibodies were purified from the supernatant by a combination of protein A affinity chromatography and size exclusion chromatography. The obtained products were characterized for identity by mass spectrometry and analytical properties such as purity by CE-SDS, monomer content and stability.

The expected primary structures were analyzed by electrospray ionization mass spectrometry (ESI-MS) of the deglycosylated intact antibody and deglycosylated/plasmin digested or alternatively deglycosylated/limited LysC digested antibody as described in the general methods section.

Additional analytical methods (e.g. thermal stability, mass spectrometry and functional assessment) were only applied after protein A and SEC purification.

### Example 2

### Determination of Binding to A131-40 Fibers In Vitro by ELISA

Binding of the bispecific antibodies to fibrillar Aβ is measured by an ELISA assay. Briefly, A13(1-40) is coated at 7 μg/mL in PBS onto Maxisorb plates for 3 days at 37° C. to produce fibrillar Abeta, and then dried for 3 h at RT. The plate is blocked with 1% CroteinC and 0.1% RSA in PBS (blocking buffer) for 1 h at RT, then washed once with wash buffer. Bispecific antibodies or controls are added at concentrations up to 100 nM in blocking buffer and incubated at 4° C. overnight. After 4 wash steps, constructs are detected by addition of anti-human-IgG-HRP (Jackson Immunoresearch) at 1:10,000 dilution in blocking buffer (1 RT), followed by 6 washes and incubation in TMB (Sigma). Absorbance is read out at 450 nm after stopping color development with 1 N HCl.

### Example 3

### Determination of Binding to Transferrin Receptor In Vitro

Binding of the bispecific antibodies to murine transferrin receptor is tested by FACS analysis on mouse X63.AG8-563 myeloma cells. If the Aβ antibody shows a certain tendency to non-specifically bind to Ag8 cells, specific binding can be quantified by co-incubation with a 20fold excess of anti-mouse-TfR antibody. Cells are harvested by centrifugation, washed once with PBS and 5×10⁴ cells incubated with a 1.5 pM to 10 nM dilution series of the polypeptide fusions with or without addition of 200 nM anti-mouse TfR antibody in 100 μL RPMI/10% FCS for 1.5 h on ice. After 2 washes with RPMI/10% FCS, cells are incubated with goat-anti-human IgG coupled to Phycoerythrin (Jackson Immunoresearch) at a dilution of 1:600 in RPMI/19% FCS for 1.5 h on ice. Cells are again washed, resuspended in RPMI/10% FCS and Phycoerythrin fluorescence measured on a FACS-Array instrument (Becton-Dickinson).

### Example 4

### Surface Plasmon Resonance-Based Binding Assay for Human TfR-Antibody Interaction

The binding experiment were carried out on a BIAcore B 4000 (GE Healthcare) equipped with C1 sensor chip (GE Healthcare, cat.no. BR1005-35) pre-treated with anti-human Fab antibody (GE Healthcare, cat.no 28-9583-25) using a standard amine coupling chemistry procedure accordingly to the vendor's manual.

For kinetic measurements the sample antibody was immobilized applying a contact time of 60 seconds and a flow rate of 10 μL/min in phosphate buffer saline pH 7.4, 0.05% Tween 20 at 25° C. Recombinant His6-tagged human transferrin receptor (“His6” disclosed as SEQ ID NO: 2) (R&D systems, cat.no 2474-TR-050) was applied in increasing concentrations and the signal monitored over the time. An average time span of 150 seconds of association time and 600 seconds of dissociation time at 30 μL/min flow rate was recorded. Data were fit using a 1:1 binding model (Langmuir isotherm).

### Example 5

Staining of Native Human β-Amyloid Plaques from Brain Sections of an Alzheimer's Disease Patient by Indirect Immunofluorescence Using a Bispecific Antibody as Produced in the Method as Reported Herein

The bispecific antibodies can be tested for the ability to stain native human 3-amyloid plaques by immunohistochemistry analysis using indirect immunofluorescence. Specific and sensitive staining of genuine human 3-amyloid plaques can be demonstrated. Cryostat sections of unfixed tissue from the temporal cortex obtained postmortem from patients positively diagnosed for Alzheimer's disease are labeled by indirect immunofluorescence. A two-step incubation is used to detect bound bispecific antibody, which is revealed by affinity-purified goat anti-human (GAH555) IgG (H+L) conjugated to Alexa 555 dye (Molecular Probes). Controls can include unrelated human IgG1 antibodies (Sigma) and the secondary antibody alone, which all should give negative results.

### Example 6

### In Vivo β-Amyloid Plaque Decoration by a Bispecific Antibody as Produced in the Method as Reported Herein in a Mouse Model of Alzheimer's Disease

Bispecific antibody can be tested in APP/PS2 double transgenic mice, a mouse model for AD-related amyloidosis (Richards, J. Neuroscience, 23 (2003) 8989-9003) for their ability to immuno-decorate β-amyloid plaques in vivo. This enabled assessment of the extent of brain penetration and binding to amyloid-β plaques. The fusion polypeptide can be administered at different doses compared to naked anti-Aβ monoclonal antibody and after 6 days animals are perfused with phosphate-buffered saline and the brains frozen on dry ice and prepared for cryosectioning.

The presence of the antibodies bound to 3-amyloid plaques can be assessed using unfixed cryostat sections either by single-labeled indirect immunofluorescence with goat anti-human IgG (H+L) conjugated to Alexa555 dye (GAH555) (Molecular Probes) at a concentration of 15 μg/ml for 1 hour at room temperature. A counterstaining for amyloid plaques can be done by incubation with BAP-2, a mouse monoclonal antibody against Aβ conjugated to Alexa 488 at a concentration of 0.5 μg/ml for 1 hour at room temperature. Slides are embedded with fluorescence mounting medium (S3023 Dako) and imaging is done by confocal laser microscopy.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 7

Transfection of Stable Cell Line Expressing Bispecific Anti-DR5/FAP Antibody with Expression Vector Comprising an Expression Cassette for the Domain Exchanged Light Chain

Clone 0131 cells were transfected with the CrossLC expression vector comprising one expression cassette for the light chain with domain crossover. Transfections were performed using linearized DNA in chemically defined medium using nucleofection (Amaxa) and 0.6/1.2/2.4 pM (total) plasmid, leading to 2×3 cell pools which were selected differently.

The transfected clone pools were selected in chemically defined medium supplemented with 10 mmol/L glutamine and by 250 nM MTX (for DHFR) plus 500 nM and 700 nM Hygromycin B. After three weeks the pools were analyzed by CE-SDS and HIC for reduction of side peaks and increase of main peak.

Based on these results the three pools which had been selected by 250 nM MTX and 700 NM HygB (0314, 0316, 0318) were chosen for Limited Dilution (LD) and plating of each 3×384w plates with chemically defined medium supplemented with 10 mmol/L glutamine and a MTX-concentration of 250 nmol/L and 700 nM HygB.

One week later the supernatants of the 3×384w plates were tested for binding to DR5 and FAP by ELISA and a DR5-FAP bridging ELISA. 158 clones with good titers and high reactivity against both antigens were picked and expanded via 24-well plates to 6 wells, where they were evaluated in a four day batch experiment (‘seed train titer’) with regard to target binding by ELISA and bridging ELISA, growth, productivity and side-product profile assessed by CE-SDS. 46 clones thereof with titers up to 830 μg/ml and acceptable product quality were further characterized in 14 days fed-batch cultures in Ambr15 system and analyzed concerning target-binding, growth properties, and side-product profile by CE-SDS and HIC. 20 clones were selected thereof and further tested by mass spectrometry (MS). 10 clones thereof selected were cultivated in shake flasks in chemically defined medium and deposited as PSBs.

1. A method for producing a multispecific antibody, which comprises at least three different polypeptides, comprising the following steps:
cultivating a mammalian cell in a cultivation medium, whereby the mammalian cell has been generated by
a) transfecting a mammalian cell with a first expression vector and one, two or three further expression vectors,
wherein the first expression vector comprises exactly one nucleic acid sequence encoding a polypeptide of the multispecific antibody, and the one, two or three further expression vectors each comprise at least two nucleic acid sequences each encoding different polypeptide chains of the multispecific antibody,
wherein the exactly one nucleic acid sequence of the first expression vector is a nucleic acid sequence encoding a light chain polypeptide of the multispecific antibody,
recovering the multispecific antibody from the cell or the cultivation medium, and thereby producing the multispecific antibody.

2. The method according to claim 1, wherein two of the polypeptide chains of the multispecific antibody comprise a domain exchange.

3. The method according to claim 2, wherein the exactly one nucleic acid of the first expression vector encodes a light chain polypeptide with domain exchange of the multispecific antibody.

4. The method according to claim 1, wherein step a) is co-transfecting the first expression vector and the one, two or three further expression vectors.

5. The method according to claim 1, wherein the mammalian cell is transfected first with the one, two or three further expression vectors and transfected thereafter with the first expression vector.

6. The method according to claim 1, wherein the mammalian cell stably expresses the multispecific antibody.

7. The method according to claim 1, wherein the mammalian cell is a CHO cell.

8. The method according to claim 2, wherein the domain exchange is a CH1-CL crossover or a VH-VL-crossover.

9. The method according to claim 1, wherein the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

10. The method according to claim 1, wherein the multispecific antibody is a bivalent, bispecific antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

11. The method according to claim 1, wherein the multispecific antibody is a trivalent, bispecific antibody, comprising
a) a first light chain and a first heavy chain of a full length antibody which specifically binds to a first antigen,
b) a second heavy chain of a full length antibody which when paired with the first light chain, specifically binds to the first antigen, and
c) a Fab fragment, which specifically binds to a second antigen, fused via a peptidic linker to the C-terminus of one of the heavy chains of a) or b), wherein the constant domains CL and CH1 of the second light chain and the second heavy chain are replaced by each other.

12. The method according to claim 1, wherein the method is for producing a multispecific antibody preparation with low/reduced product-related impurities.


SEQUENCE LISTING (2 sequences)

SEQ ID NO: 1 - protein, 20 aa, Artificial Sequence
  Description of Artificial Sequence Synthetic peptide
GGGGSGGGGSGGGGSGGGGS

SEQ ID NO: 2 - protein, 6 aa, Artificial Sequence
  Description of Artificial Sequence Synthetic 6xHis tag
HHHHHH